(19) United States
(12) Patent Application Publication
(10) Pub. No.: US 2025/0352253 A1
(43) Pub. Date: Nov. 20, 2025

(54) ORTHOPEDIC SCREW AND DRIVER SYSTEM FOR MINIMALLY INVASIVE METATARSAL CORRECTION PROCEDURE

(71) Applicant: Treace Medical Concepts, Inc., Ponte Vedra, FL (US)

(72) Inventors: Adriaan Kuyler, Ponte Vedra, FL (US); Jason May, St. John's, FL (US); Caitlin DiGeorgio, Jacksonville, FL (US); Sean F. Scanlan, Jacksonville, FL (US); Jonathan M. Dewey, Ponte Vedra, FL (US)

(21) Appl. No.: 19/212,464

(22) Filed: May 19, 2025

Related U.S. Application Data
(60) Provisional application No. 63/649,208, filed on May 17, 2024.

(51) Int. Cl. A61B 17/86

(52) U.S. Cl. CPC A61B 17/8615

(57) ABSTRACT

A screw for osteosynthesis includes a body, a thread, and a driver receiving cavity. The body includes a proximal end and a distal end, and the body defines a central longitudinal axis that extends between the proximal end and the distal end. The thread is disposed along at least a portion of the body. The driver receiving cavity is at the proximal end of the body, and the driver receiving cavity includes at least five lobes circumferentially spaced apart from one another about the central longitudinal axis to define at least five grooves. Each one of the at least five lobes defines a different internal lobular area. The at least five grooves can be configured to receive six lobes of a hexalobular driver having equally spaced lobes.

### CROSS-REFERENCE

This application claims the benefit of U.S. Provisional Application No. 63/649,208, filed May 17, 2024, the entire contents of which are incorporated herein by reference.

### TECHNICAL FIELD

This disclosure relates to orthopedic screw and driver devices, systems, and surgical techniques.

### BACKGROUND

Bones within the human body, such as bones in the foot, may be anatomically misaligned. For example, one common type of bone deformity is hallux valgus, which is a progressive foot deformity in which the first metatarsophalangeal joint is affected and is often accompanied by significant functional disability and foot pain. The metatarsophalangeal joint is laterally deviated, resulting in an abduction of the first metatarsal while the phalanges adduct. This often leads to development of soft tissue and a bony prominence on the medial side of the foot, which is called a bunion.

Surgical intervention may be used to correct a bunion deformity. A variety of different surgical procedures exist to correct bunion deformities and may involve removing the abnormal bony enlargement on the first metatarsal and/or attempting to realign the first metatarsal relative to the adjacent metatarsal. In some applications, an osteotomy is performed that involves cutting the metatarsal into two portions and shifting the cut distal portion medially to reduce the prominence of the bunion. The repositioned one or more portions of the metatarsal can then be fixated using a bone fixation device. Surgical instruments that can facilitate efficient and consistent bone fixation device implantation are useful for practitioners performing osteosynthesis and other bone realignment and fixation techniques.

### SUMMARY

In general, this disclosure is directed to orthopedic screw and driver devices, systems, and surgical techniques for performing an osteosynthesis, such as at one or more bones of a foot. The described devices, systems, and techniques can be utilized to partially or fully fixate a corrected anatomical alignment of the one or more bones. Example instruments and techniques described in the present disclosure can be used to fixate a moved position of one bone portion relative to another bone portion.

In various example techniques disclosed herein, a clinician can surgically access a bone and cut the bone into at least two portions: a distal portion which may be referred to as a capital fragment and a proximal portion. With the bone cut into two portions, the clinician can move the distal portion relative to the proximal portion, e.g., to help correct an anatomical deformity. For example, the clinician can shift the distal portion in the transverse plane (e.g., move the distal portion laterally), rotate the distal portion in the frontal plane, and/or shift the distal portion in the sagittal plane. In some implementations, the clinician engages the distal portion with a bone positioning device operable to controllably move the distal portion relative to the proximal portion. Before, after, and/or while moving the distal portion of the cut bone relative to the proximal portion in one or more planes, the clinician may implant, at the proximal and/or distal bone portions, a screw for osteosynthesis as will be disclosed herein.

Screw and/or driver systems according to the disclosure can provide a variety of advantages for the clinician and patient. In some configurations, for instance, the screw may include a plurality of differently sized lobes that are arranged and/or indexed relative to an alignment feature of the screw. The alignment feature can be configured to align with a predetermined anatomical feature of a patient, such as a predetermined anatomical feature defined at the foot of the patient associated with a minimally invasive osteosynthesis procedure (e.g., an outer surface of a metatarsal bone portion of the foot). The driver may also include an alignment feature that is positioned at a specific orientation relative to the alignment feature of the screw (when the driver is engaged with the lobes of the screw in an orientation set by the configuration of the different lobes). During the surgical procedure, the clinician can manipulate the position of the alignment feature of the driver (e.g., by controlling the number and/or extent of rotation of the driver) to control the position of the corresponding alignment feature of the screw. For a minimally invasive procedure where the screw is inserted through a small opening in the skin (e.g., percutaneous poke hole or small incision), this arrangement can allow the clinician to visualize and control the positioning of the screw alignment feature even though the clinician may have limited or no visibility of the screw under the skin.

A screw for osteosynthesis as disclosed herein can provide a number of additional or alternative advantages. As one example, various embodiments of the screw for osteosynthesis as disclosed herein can include a plurality of differently sized lobes (e.g., three or more; four or more; five or more; six or more). The plurality of differently sized lobes at the screw can act to create additional contact driving interference between a driver and the plurality of differently sized lobes at the screw as compared to a screw that has multiple same-sized lobes for driver engagement and torque transfer. This added contact driving interference with a driver so configured can help to prevent driver slip out relative to the screw yet while providing sufficient torque force transfer from the driver to the screw. In addition, the plurality of differently sized lobes at the screw can be configured to engage both a screw-specific driver and a hexalobular driver that has equally spaced lobes. This can configure the screw to be both implanted and removed using a screw-specific driver and to be both implanted and removed using a more standard hexalobular driver. In this way, the screw can be configured for use with both a more standard hexalobular driver but also for use with a screw-specific driver that can, for instance, facilitate more tailored screw-specific implantation using the screw-specific driver.

In one example, a screw for osteosynthesis is described. This screw embodiment includes a body, a thread, and a driver receiving cavity. The body includes a proximal end and a distal end, and the body defines a central longitudinal axis that extends between the proximal end and the distal end. The thread is disposed along at least a portion of the body. The driver receiving cavity is at the proximal end of the body, and the driver receiving cavity includes at least five lobes circumferentially spaced apart from one another about the central longitudinal axis to define at least five grooves. Each one of the at least five lobes defines a different internal lobular area. The at least five grooves may or may not be configured to receive six lobes of a hexalobular driver having equally spaced lobes.

In a further embodiment of this screw, the screw also includes an alignment feature disposed along at least a portion of the body. The at least five lobes can be indexed relative to the alignment feature. In one example, the body has a circular cross-sectional shape, and the alignment feature includes a bevel extending along at least a portion of a length of the body.

In a further embodiment of this screw, one of the at least five grooves is configured to receive two lobes of the hexalobular driver.

In a further embodiment of this screw, one or more of the at least five lobes is configured to engage the hexalobular driver in a first hexalobular driver rotational direction corresponding to removal of the screw, and one or more of the at least five lobes is configured to engage the hexalobular driver in a second hexalobular driver rotational direction corresponding to advancement of the screw, with the second hexalobular driver rotational direction being opposite the first hexalobular driver rotational direction. For example, at least two of the at least five lobes can be configured to engage the hexalobular driver in the first hexalobular driver rotational direction, and at least two of the at least five lobes are configured to engage the hexalobular driver in the second hexalobular driver rotational direction. As another additional or alternative example, a different number of the at least five lobes can be configured to engage the hexalobular driver in the first hexalobular driver rotational direction than in the second hexalobular driver rotational direction.

In a further embodiment of this screw, the driving receiving cavity defines a sidewall, and each of the at least five lobes extends from the sidewall to an innermost projecting surface. The internal lobular area of each of the at least five lobes can be defined by a region extending from the sidewall to the innermost projecting surface. In some embodiments, the internal lobular area of one of the at least five lobes can be at least twice as large as the internal lobular area of at least one other of the at least five lobes.

In a further embodiment of this screw, each of the at least five grooves defines a different area than each other of the at least five grooves.

In a further embodiment of this screw, the at least five lobes are only five lobes, and the at least five grooves is only five grooves.

In a further embodiment of this screw, the thread disposed along at least a portion of the body includes a first threaded region configured to be inserted into a first bone portion and a second threaded region configured to be inserted into a second bone portion. The first threaded region can be separated from the second threaded region by a transition region defining at least one cutting feature. The first threaded region can extend from the distal end to the transition region, and the second threaded region can extend from the proximal end to the transition region. As one example, the at least one cutting feature at that transition region can include a plurality of cutting flutes spaced about a perimeter of the transition region.

In a further embodiment of this screw, the distal end includes a plurality of cutting teeth that extend axially outwardly.

In a further embodiment of this screw, the driver receiving cavity includes a driver receptacle. The driver receptacle extends from the proximal end of the body toward the distal end of the body. The at least five lobes can be spaced distally from the proximal end, and each of the at least five lobes can include at least two lobular sidewalls that protrude radially inwardly into the driver receptacle. In a still further embodiment, the screw can also include a driver nub receiving bore defined at the body and extending along the central longitudinal axis. The driver nub receiving bore can be spaced distally from the proximal end and distally from the at least five lobes, The driver nub receiving bore can define a first internal body diameter transverse to the central longitudinal axis, and each of the at least five lobes can define a second internal body diameter transverse to the central longitudinal axis. The first internal body diameter can be smaller than the second internal body diameter.

In a further embodiment of this screw, the body can have a length extending from the proximal end to the distal end sized to be positioned across two portions of a metatarsal bone.

In another example, a system is described. This system embodiment includes an osteosynthesis screw and a screw-specific driver. The osteosynthesis screw includes a body, a thread, and a driver receiving cavity. The body that includes a proximal end and a distal end. The body defines a screw central longitudinal axis that extends between the proximal end and the distal end. The thread is disposed along at least a portion of the body. The driver receiving cavity is at the proximal end of the body. The driver receiving cavity includes at least three lobes circumferentially spaced apart from one another about the screw central longitudinal axis to define at least three grooves. Each one of the at least three lobes defines a different internal lobular area. The at least three grooves may be configured to receive a hexalobular driver having equally spaced lobes. The screw-specific driver includes a driver body and at least three driving lobes at the driver body. The driver body includes a proximal end and a distal end, and the driver body defines a driver central longitudinal axis. The at least three driving lobes are circumferentially spaced apart from one another about the driver central longitudinal axis. Each one of the at least three driving lobes defines a different driving lobular area, and each one of the at least three different driving lobular area correspond to one of the at least three grooves.

In a further embodiment of this system, the osteosynthesis screw further includes a driver nub receiving bore and the screw-specific driver further includes a driver engagement nub. The driver nub receiving bore is defined at the body and extends along the driver central longitudinal axis. The driver nub receiving bore is spaced distally from the proximal end and distally from the at least three lobes. The driver nub receiving bore defines a first internal body diameter transverse to the driver central longitudinal axis. The driver engagement nub is at the distal end of the driver body. The at least three driving lobes are spaced proximally from the driver engagement nub such that the driver engagement nub is configured to engage the osteosynthesis screw prior to the at least three driving lobes engaging the osteosynthesis screw.

In a further embodiment of this system, the driver engagement nub defines a first external driver body diameter transverse to the driver central longitudinal axis, and each of the at least three driving lobes defines a second external driver body diameter transverse to the driver central longitudinal axis, where the first external driver body diameter is smaller than the second external driver body diameter.

In a further embodiment of this system, the osteosynthesis screw further includes an alignment feature disposed along at least a portion of the body, and the at least three lobes are indexed relative to the alignment feature. For example, the screw body can have a circular cross-sectional shape, and the alignment feature can include a bevel extending along at least a portion of a length of the body.

In a further embodiment of this system, one of the at least three grooves is configured to receive two lobes of the hexalobular driver.

In a further embodiment of this system, each of the at least three grooves defines a different area than each other of the at least three grooves.

In a further embodiment of this system, the osteosynthesis screw has five lobes and five grooves, and each of the five grooves defines a different area than each other of the five grooves.

In a further embodiment of this system, one or more of the at least three lobes is configured to engage the hexalobular driver in a first hexalobular driver rotational direction corresponding to removal of the screw, and one or more of the at least three lobes is configured to engage the hexalobular driver in a second hexalobular driver rotational direction corresponding to advancement of the screw, the second hexalobular driver rotational direction being opposite the first hexalobular driver rotational direction. For example, at least two of the at least three lobes can be configured to engage the hexalobular driver in the first hexalobular driver rotational direction, and at least two of the at least three lobes can be configured to engage the hexalobular driver in the second hexalobular driver rotational direction. As an additional or alternative example, a different number of the at least three lobes can be configured to engage the hexalobular driver in the first hexalobular driver rotational direction than in the second hexalobular driver rotational direction.

Another embodiment includes a bone fixation technique. This technique embodiment includes: rotationally driving an osteosynthesis screw attached to a screw-specific driver through a first bone portion and into a second bone portion and across a separation between the first bone portion and the second bone portion. The screw-specific driver is engaged with a driver receiving cavity of the osteosynthesis screw. The driver receiving cavity includes at least three lobes circumferentially spaced apart from one another about a screw central longitudinal axis to define at least three grooves, with each one of the at least three lobes defining a different internal lobular area. The at least three grooves may be configured to receive a hexalobular driver having equally spaced lobes that is different than the screw-specific driver having asymmetrically sized and/or spaced lobes.

In some implementations, this technique may include making an incision through a skin of a patient and retracting the skin along the incision to expose one or both of the first bone portion and the second bone portion. In additional or alternative implementations, the technique can be a percutaneous technique (e.g., utilizing a poke hole through the skin) without an incision to expose the target bone portion(s).

In a further embodiment, this technique additionally includes cutting a bone into the first bone portion and the second bone portion. As one example, the bone can be a metatarsal.

In a further embodiment, this technique additionally includes, prior to rotationally driving the osteosynthesis screw, moving the first bone portion relative to the second bone portion.

In a further embodiment, this technique additionally includes attaching the osteosynthesis screw to the screw-specific driver.

In a further embodiment, this technique additionally includes, after rotationally driving the osteosynthesis screw, disengaging the screw-specific driver from the osteosynthesis screw by disengaging at least three driving lobes of the screw-specific driver from the at least three grooves of the osteosynthesis screw.

In a further embodiment, this technique additionally includes, after rotationally driving the osteosynthesis screw and disengaging the screw-specific driver from the osteosynthesis screw, attaching the hexalobular driver to the osteosynthesis screw and removing the osteosynthesis screw by rotationally driving the hexalobular driver.

The details of one or more examples are set forth in the accompanying drawings and the description below. Other features, objects, and advantages will be apparent from the description and drawings, and from the claims.

### BRIEF DESCRIPTION OF DRAWINGS

FIGS. 1A and 1B are front views of a foot showing a normal first metatarsal position and an example frontal plane rotational misalignment position, respectively.

FIGS. 2A and 2B are top views of a foot showing a normal first metatarsal position and an example transverse plane misalignment position, respectively.

FIGS. 3A and 3B are side views of a foot showing a normal first metatarsal position and an example sagittal plane misalignment position, respectively.

FIGS. 4A and 4B illustrate an embodiment of a screw. FIG. 4A is a side elevational view of the screw, and FIG. 4B is a perspective view of an end of the screw with a driver receiving cavity.

FIG. 5 is a perspective view of an embodiment of a screw-specific driver.

FIG. 6 is a plan view of one embodiment of a plurality of lobes that can be included on the screw of FIGS. 4A-4B.

FIGS. 7A and 7B illustrate the example lobe arrangement of FIG. 6 relative to example driving lobes of an embodiment of a screw-specific driver. FIG. 7A is a plan view of a screw-specific driver's driving lobes at a first screw-specific driver rotational position relative to the screw's plurality of lobes. FIG. 7B is a plan view of the screw-specific driver's driving lobes at a second, different screw-specific driver rotational position relative to the screw's plurality of lobes.

FIGS. 8A and 8B illustrate the example lobe arrangement of FIG. 6 relative to example driving lobes of a hexalobular driver. FIG. 8A is a plan view of the screw's plurality of lobes engaging the hexalobular driver's driving lobes in a first hexalobular driver rotational direction. FIG. 8B is a plan view of the screw's plurality of lobes engaging the hexalobular driver's driving lobes in a second, opposite hexalobular driver rotational direction.

FIGS. 9A and 9B illustrate another embodiment of a plurality of lobes that can be used with the screw of FIGS. 4A-4B. FIG. 9A is a plan view of the screw's plurality of lobes engaging the hexalobular driver's driving lobes in a first hexalobular driver rotational direction. FIG. 9B is a plan view of the screw's plurality of lobes engaging the hexalobular driver's driving lobes in a second, opposite hexalobular driver rotational direction.

FIG. 10 is a flow diagram of an example a bone fixation technique.

FIGS. 11A-11D illustrate an exemplary sequence of implanting one or more screws through a first bone portion, into a second bone portion, and across a separation between the first and second bone portions.

FIGS. 12A and 12B illustrate an embodiment of a screw-specific driver, which includes an alignment feature locational indicator, coupled to a screw, which includes an alignment feature. FIG. 12A shows a perspective view of this coupling at a first side, and FIG. 12B shows an elevational view of this coupling at a second, different side.

FIGS. 13A and 13B are a side view and an end view, respectively, of an example configuration of a screw configured as a variable pitch fully threaded screw with a beveled head.

FIGS. 14A and 14B are a side view and an end view, respectively, of an example configuration of a screw configured as partially threaded screw for an osteotomy procedure.

FIGS. 15A and 15B are a side view and an end view, respectively, of an example configuration of a screw configured as variable pitch fully threaded cortical screw.

FIGS. 16A and 16B are a side view and an end view, respectively, of an example configuration of a screw configured as variable pitch fully threaded cortical screw with a beveled head.

FIGS. 17A and 17B are a side view and an end view, respectively, of an example configuration of a screw configured as variable pitch partially threaded cortical screw with a beveled head.

FIGS. 18A and 18B are a side view and an end view, respectively, of an example configuration of a screw configured as variable pitch partially threaded cortical screw.

FIGS. 19A and 19B are a side view and an end view, respectively, of an example configuration of a screw configured as variable pitch partially threaded headless screw with a beveled head/end.

FIGS. 20A and 20B are a side view and an end view, respectively, of an example configuration of a screw configured as variable pitch fully threaded headless screw with a beveled head/end.

### DETAILED DESCRIPTION

This disclosure generally relates to bone fixation devices, systems, and techniques, particularly bone screw and driver devices, systems, and techniques. The described bone screw and driver configurations can be used in connection with a bone osteotomy and realignment procedure in which a bone is cut into at least two portions and one portion is moved relative to another portion. In an exemplary application, the devices and techniques can be used during a surgical procedure performed on one or more bones, such as bones in the foot or hand, where the bones are relatively small compared to bones in other parts of the human anatomy. In one example, a procedure utilizing embodiments of the disclosure can be performed to correct metatarsal misalignment. An example of such a procedure is a bunion correction procedure where an osteotomy is performed on a first metatarsal of the foot to divide the first metatarsal into a proximal portion and a distal portion. The distal portion of the first metatarsal can be moved (e.g., laterally) relative to the proximal portion to reduce or eliminate the boney prominence of the bunion. Another example is a bunionette correction procedure (also known as a tailor's bunion procedure) performed on a fifth metatarsal of the foot to divide the fifth metatarsal into a proximal portion and a distal portion. The distal portion of the fifth metatarsal can be moved (e.g., medially) relative to the proximal portion to reduce or eliminate the boney prominence on the fifth metatarsal.

While techniques and devices are generally described herein in connection with different portions of a single metatarsal (e.g., distal and proximal portions of a first metatarsal of the foot), the techniques and devices may be used on other adjacent bones (e.g., separated from each other by a joint) and/or adjacent bone portions (e.g., portions of the same bone separated from each other by a fracture or osteotomy). In various examples, the devices, systems, and/or techniques of the disclosure may be utilized on comparatively small bones in the foot such as a metatarsal (e.g., first, second, third, fourth, or fifth metatarsal), a cuneiform (e.g., medial, intermediate, lateral), a cuboid, a phalanx (e.g., proximal, intermediate, distal), and/or combinations thereof. The bones may be separated from each other by a tarsometatarsal (“TMT”) joint, a metatarsophalangeal (“MTP”) joint, or other joint or osteotomy. Accordingly, reference to a distal metatarsal portion and a proximal metatarsal portion herein may be replaced with other bone pairs as described herein. Further, where an implant (e.g., screw) according to the disclosure is intended to be used on a different bone or combination of bones other than different portions of the first metatarsal, the configuration of the implant (e.g., size, shape) may be adjusted to accommodate the specific bone or combination of bones being fixated while following the configuration teachings outlined herein.

For example, the bone screw and/or driver configurations described herein can be used to fixate any first bone portion with any second bone portion, where the first bone portion is separated from the second bone portion by a joint, fracture, and/or osteotomy. Accordingly, while specific anatomical applications may be described herein, the described bone screw and/or driver configurations may be used to fixate other first and second bone portions (with the screw extending from the first bone portion to the second bone portion), wherein the first and second bone portions are different bones or different portions of a same bone.

In some examples, an osteotomy procedure is performed to treat hallux valgus, which is referred to as a bunion. Hallux valgus, also referred to as hallux abducto valgus, is a complex progressive condition that is characterized by lateral deviation (valgus, abduction) of the hallux and medial deviation of the first metatarsophalangeal joint. Hallux valgus typically results in a progressive increase in the hallux abductus angle, the angle between the long axes of the first metatarsal and proximal phalanx in the transverse plane. An increase in the hallux abductus angle may tend to laterally displace the plantar aponeurosis and tendons of the intrinsic and extrinsic muscles that cross over the first metatarsophalangeal joint from the metatarsal to the hallux. Consequently, the sesamoid bones may also be displaced, e.g., laterally relative to the first metatarsophalangeal joint, resulting in subluxation of the joints between the sesamoid bones and the head of the first metatarsal. This can increase the pressure between the medial sesamoid and the crista of the first metatarsal head.

In some examples, an osteotomy procedure is performed to treat a tailor's bunion, also known as digitus quintus varus or bunionette. A bunionette is a callus and an adventitious bursa that overlies a prominent, laterally deviated fifth metatarsal head and a medially deviated fifth toe.

While devices and techniques are generally described herein in connection with the first metatarsal of the foot as part of a bunion correction procedure, the techniques and devices may be used on other bones and/or to treat other bone conditions. In various examples, the devices, systems, and/or techniques of the disclosure may be utilized on comparatively small bones in the foot such as a metatarsal (e.g., first, second, third, fourth, or fifth metatarsal), a cuneiform (e.g., medial, intermediate, lateral), a cuboid, a phalanx (e.g., proximal, intermediate, distal), and/or combinations thereof.

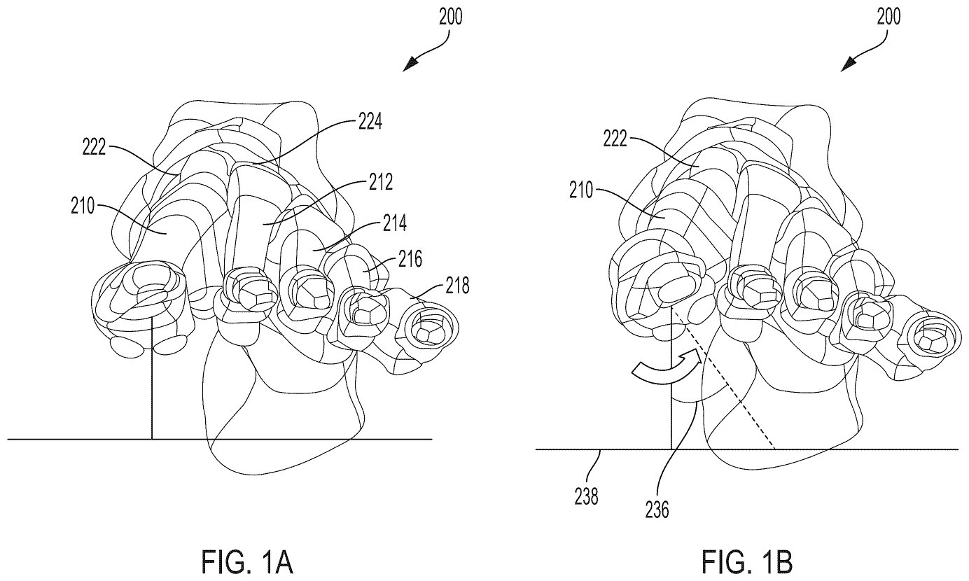

To further understand example techniques of the disclosure, the anatomy of the foot will first be described with respect to FIGS. 1-3 along with example misalignments that may occur and be corrected according to the present disclosure. A bone misalignment may be caused by hallux valgus (bunion), bunionette, a natural growth deformity, and/or other condition.

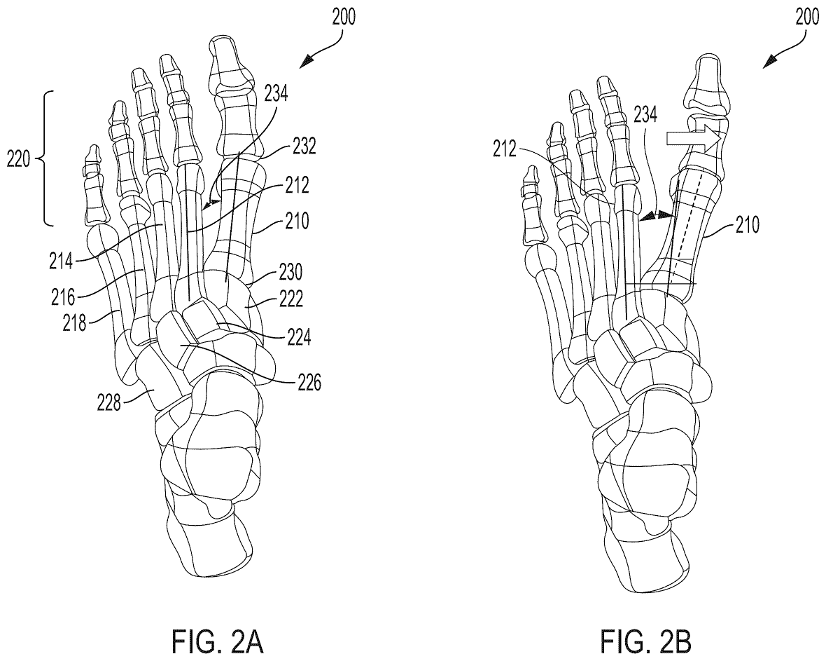

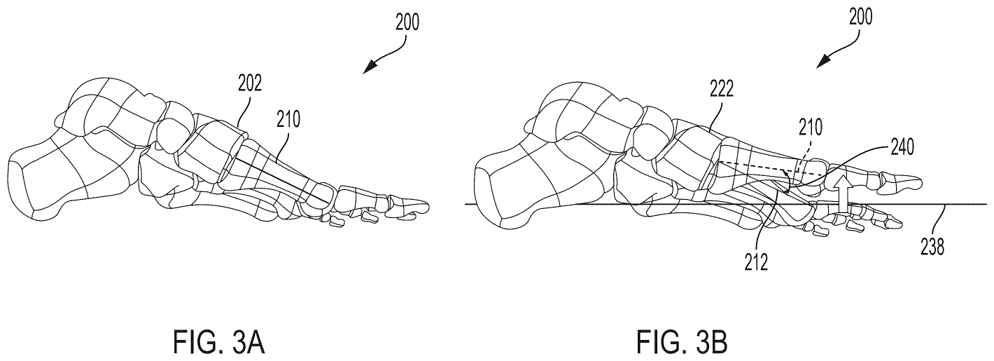

FIGS. 1A and 1B are front views of foot 200 showing a normal first metatarsal position and an example frontal plane rotational misalignment position, respectively. FIGS. 2A and 2B are top views of foot 200 showing a normal first metatarsal position and an example transverse plane misalignment position, respectively. FIGS. 3A and 3B are side views of foot 200 showing a normal first metatarsal position and an example sagittal plane misalignment position, respectively. While FIGS. 1B, 2B, and 3B show each respective planar misalignment in isolation, in practice, a metatarsal may be misaligned in any two of the three planes or even all three planes. Accordingly, it should be appreciated that the depiction of a single plane misalignment in each of FIGS. 1B, 2B, and 3B is for purposes of illustration and a metatarsal may be misaligned in multiple planes that is desirably corrected. Further, a bone condition treated according to the disclosure may not present any of the example misalignments described with respect to FIGS. 1B, 2B, and 3B, and it should be appreciated that the disclosure is not limited in this respect.

With reference to FIGS. 1A and 2A, foot 200 is composed of multiple bones including a first metatarsal 210, a second metatarsal 212, a third metatarsal 214, a fourth metatarsal 216, and a fifth metatarsal 218. The metatarsals are connected distally to phalanges 220 and, more particularly, each to a respective proximal phalanx. The first metatarsal 210 is connected proximally to a medial cuneiform 222, while the second metatarsal 212 is connected proximally to an intermediate cuneiform 224 and the third metatarsal is connected proximally to lateral cuneiform 226. The fourth and fifth metatarsals 216, 218 are connected proximally to the cuboid bone 228. The joint 230 between a metatarsal and respective cuneiform (e.g., first metatarsal 210 and medial cuneiform 222) is referred to as the tarsometatarsal (“TMT”) joint. The MTP joint 232 between a metatarsal and respective proximal phalanx is referred to as a metatarsophalangeal (“MTP”) joint. The angle 234 between adjacent metatarsals (e.g., first metatarsal 210 and second metatarsal 212) is referred to as the intermetatarsal angle (“IMA”).

As noted, FIG. 1A is a frontal plane view of foot 200 showing a typical position for first metatarsal 210. The frontal plane, which is also known as the coronal plane, is generally considered any vertical plane that divides the body into anterior and posterior sections. On foot 200, the frontal plane is a plane that extends vertically and is perpendicular to an axis extending proximally to distally along the length of the foot. FIG. 1A shows first metatarsal 210 in a typical rotational position in the frontal plane. FIG. 1B shows first metatarsal 210 with a frontal plane rotational deformity characterized by a rotational angle 236 relative to ground, as indicated by line 238.

FIG. 2A is a top view of foot 200 showing a typical position of first metatarsal 210 in the transverse plane. The transverse plane, which is also known as the horizontal plane, axial plane, or transaxial plane, is considered any plane that divides the body into superior and inferior parts. On foot 200, the transverse plane is a plane that extends horizontally and is perpendicular to an axis extending dorsally to plantarly (top to bottom) across the foot. FIG. 2A shows first metatarsal 210 with a typical IMA 234 in the transverse plane. FIG. 2B shows first metatarsal 210 with a transverse plane rotational deformity characterized by a greater IMA caused by the distal end of first metatarsal 210 being pivoted medially relative to the second metatarsal 212.

FIG. 3A is a side view of foot 200 showing a typical position of first metatarsal 210 in the sagittal plane. The sagittal plane is a plane parallel to the sagittal suture which divides the body into right and left halves. On foot 200, the sagittal plane is a plane that extends vertically and is perpendicular to an axis extending proximally to distally along the length of the foot. FIG. 3A shows first metatarsal 210 with a typical rotational position in the sagittal plane. FIG. 3B shows first metatarsal 210 with a sagittal plane rotational deformity characterized by a rotational angle 240 relative to ground, as indicated by line 238.

Surgical techniques and instruments according to the disclosure can be useful to treat a misalignment of one or more bones of the foot, such as first metatarsal 210. In some applications, a technique involves surgically accessing first metatarsal 210. The clinician may utilize an incision guide to identify the location and size of the incision to be made relative to first metatarsal 210 prior to making the incision through the skin of the patient to surgically access the bone. After making the incision through the skin of the patient, the clinician may attach a cutting guide, also referred to as a bone preparation guide, having one or more guide surfaces configured to guide a cutting instrument. The clinician can use the cutting guide to guide the cutting instrument to cut the first metatarsal into a distal portion (which can be referred to as a capital fragment) and a residual proximal portion.

With the first metatarsal cut into two portions, the distal portion can be realigned in one or more planes relative to the proximal portion to reduce or eliminate an anatomic misalignment. For example, the distal portion can be realigned in two or more planes, or three planes relative to the proximal portion. In some examples, the distal portion is moved laterally in a transverse plane relative to the proximal portion (e.g., to reduce the bony prominence associated with the bunion deformity), the distal portion is rotated in a frontal plane relative to the proximal portion (e.g., to reposition the sesamoid bones dorsally under the distal portion), and/or the distal portion is plantar flexed or dorsiflexed in the sagittal plane. The repositioning of the distal portion can occur via the clinician's hand (e.g., grasping one or more wires inserted into the distal portion) and/or with the aid of instrumentation that applies a force in one or more planes to control repositioning of the distal portion. The clinician can install a fixation device across the osteotomy location between the distal portion and proximal portion to fixate a moved position of the distal portion relative to the proximal portion. The fixation device can hold the moved position of the distal portion to allow bone to form and grow between the proximal portion and moved distal portion, thereby fusing the two portions together. The clinician may utilize one or more instruments, implants, and/or techniques according to disclosure to perform the osteotomy, bone realignment, and/or fixation of the realigned bone portions.

The present disclosure describes various embodiments of a fixation device in the form of a screw and also describes embodiments of drivers that can be used with the screw to implant the screw and/or remove the screw after implantation. The screw can, for example, be configured for osteosynthesis, such as configured for osteosynthesis by implantation through a first bone portion, into a second bone portion, and across a separation between the first and second bone portions. As one exemplary application, the screw can be configured to be implanted through a first metatarsal portion, into a second metatarsal portion, and across a space that separates the first and second metatarsal portions. Reference to a separation between a first bone portion and a second bone portion can refer to a fracture location, an osteotomy location, and/or joint between two different bone portions even though opposed end portions of the two bone portions can be in contact with each other (e.g., without a space or gap between the opposed bone ends).

Various embodiments of a screw for osteosynthesis as disclosed herein can include a plurality of differently sized lobes (e.g., three or more; four or more; five or more; six or more). The plurality of differently sized lobes can function to provide additional contact surface area between a driver and the plurality of differently sized lobes at the screw (e.g., as compared to a screw that has multiple same-sized lobes for driver engagement and torque transfer). This contact driving interference with a driver corresponding to the plurality of differently sized lobes at the screw can help to prevent driver slip out relative to the screw while still providing sufficient torque force transfer from the driver to the screw. In addition, the plurality of differently sized lobes of the screw can be configured to engage both a screw-specific driver and a hexalobular driver that has equally spaced lobes. This can configure the screw to be both implanted and removed using a screw-specific driver and to be both implanted and removed using a more standard hexalobular driver for clinician convenience and cost efficiencies depending on the tools available at the time of a procedure (e.g., at the time of a procedure to remove the screw). Moreover, the plurality of differently sized lobes of the screw can be indexed relative to an alignment feature also included at the screw. As a result, the differently sized lobes can be used as an indexing feature to control positioning of the alignment feature of the screw relative to one or more bone portions. This can be particularly helpful when implanting the screw in a minimally invasive procedure, where limited or no visibility to the alignment feature may be present under or through the skin of the patient. These and other useful aspects will be appreciated in view of the following disclosure relating to various embodiments of such a screw and related systems and techniques.

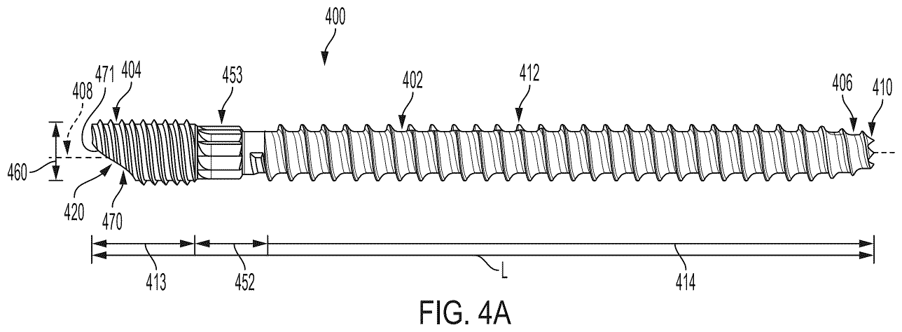

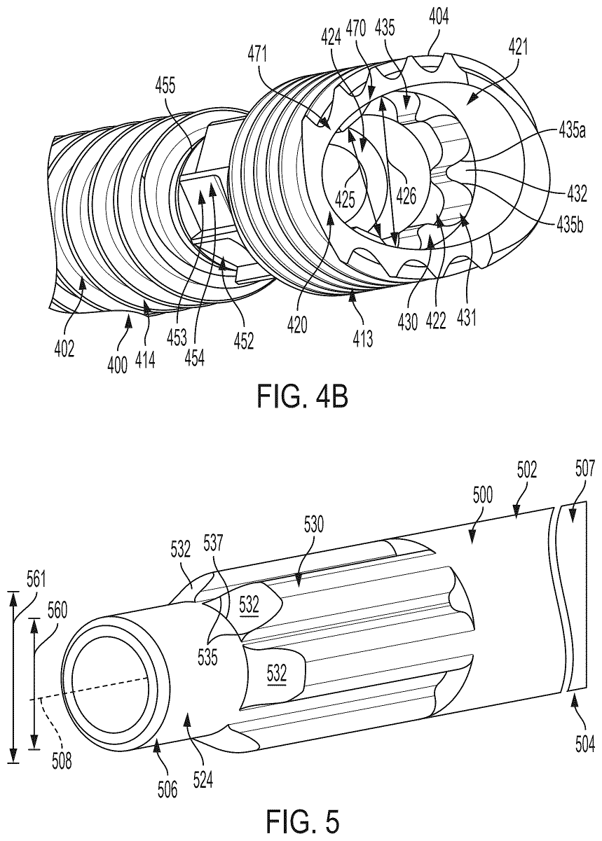

FIGS. 4A-4B illustrate an embodiment of a screw 400. In particular, FIG. 4A shows a side elevational view of the screw 400, and FIG. 4B shows a perspective view of an end of the screw 400. The screw 400 can be configured for use in an osteosynthesis technique as will be described herein, though other applications of the screw 400 are also within the scope of the present disclosure.

The screw 400 can include a body 402. The body 402 can include a proximal end 404 and a distal end 406 that is opposite the proximal end 404. The body 402 can define a central longitudinal axis 408 that extends between the proximal end 404 and the distal end 406, and the body 402 can extend a length L from the proximal end 404 to the distal end 406 (e.g., and in a direction along the central longitudinal axis 408). The body can extend the length L from the proximal end 404 to the distal end 406 such that the length L is sized to be positioned across two portions of a bone. For example, the length L of the body 402 of the screw 400 can be sized to be positioned across two portion of a metatarsal bone, such as a metatarsal bone in the foot (e.g., a first metatarsal bone in the foot). As one such particular example, the length L of the body 402 of the screw 400 can be sized to be positioned through a first bone portion (e.g., through a first metatarsal bone portion in the foot), into a second bone portion (e.g., into a second metatarsal bone portion in the foot), and across a separation between these first and second bone portions (e.g., across an osteotomy dividing a single metatarsal bone into two portions). Such length L of the body 402 of the screw 400 can, for instance, range from 15-75 mm, such as ranging from 20-60 mm, ranging from 20-50 mm, or ranging from 30-60 mm depending on the particular bones at which the screw 400 is configured for implantation.

The illustrated embodiment of the screw 400 shows that the distal end 406 of the screw 400 can include one or more cutting teeth 410. The one or more cutting teeth 410 can be configured to cut bone material as the screw 400 is driven into the bone material. The illustrated embodiment of the screw 400 includes a plurality of cutting teeth 410 that extend axially outward at the distal end 406 in a direction parallel to the central longitudinal axis 408 of the body 402. As illustrated here, the plurality of cutting teeth 410 can be circumferentially spaced apart from one another about the central longitudinal axis 408. As also illustrated here, the distal end of the plurality of cutting teeth 410 can form a distal-most surface of the body 402 and, thus, the plurality of cutting teeth 410 can be a first structure at the body 402 that contacts an outer surface of a bone portion prior to rotationally driving the screw 400. In some embodiments, the distal end 406 can, in addition to or alternative to the cutting teeth 410, include a self-tapping feature. When included, the self-tapping feature can be configured to provide a self-tapping function at an outer surface of a bone portion as the screw 400 is initially rotationally driven into that bone portion.

The screw 400 can include threading 412 disposed along at least a portion of the body 402. Threading 412 can partially or fully encircle the body 402 of the screw 400 for engaging with one or more bone portions at which the screw 400 is to be inserted. Threading 412 can be a helical structure used to convert rotational motion into linear movement or force. Threading 412 can be defined by a ridge of material wrapped around an inner cylinder or cone of material in the form of a helix, to define a straight thread or a tapered thread. In some examples, threading 412 can be self-tapping and/or body 402 can taper at the distal end 406 to facilitate ease of insertion of the screw 400 into a bone. In various examples, the screw 400 can be configured as a single-lead screw or as a multi-lead screw, such as a dual lead screw, a tri-lead screw, or a quad-lead screw, such that each time the body 402 of the screw 400 is rotated one turn, it is advanced axially by a multiple of the pitch or spacing between adjacent ridges.

When the screw 400 includes threading 412, the screw 400 can be threaded along the entire length L of the body 402 or can be threaded along less than the entire length L of the body 402. For instance, when the screw 400 has threading 412 along less than the entire length L of the body 402, such as shown for the illustrated embodiment, the screw 400 can have threading 412 disposed along different regions of the body 402. In particular, the illustrated embodiment of the screw 400 includes a transition region 452, along a length of the body 402, that lacks threading 412, while the illustrated embodiment of screw 400 also includes a first threaded region 413 along a length of the body 402 and a second threaded region 414 along a length of the body 402. As shown here, the first threaded region 413 can be separated from the second threaded region 414 by the transition region 452. For instance, the first threaded region 413 can extend from the proximal end 404 to the transition region 452, and the second threaded region 414 can extend from the distal end 406 to the transition region 452.

In use, for example, the first threaded region 413 of the screw 400 can be configured to be inserted into a first bone portion and the second threaded region 414 of the screw 400 can be configured to be inserted into a second bone portion, and the transition region 452 can be configured to be positioned at the first bone portion, at the second bone portion, and/or at a space between the first and second bone portions at which the first and second threaded regions 413, 414 are positioned. As one specific such example, the first threaded region 413 can be configured to be inserted into a first metatarsal portion and the second threaded region 414 can be configured to be inserted into a second metatarsal portion, and the transition region 452 can be configured to be positioned at least at a space between the first and second metatarsal portions at which the first and second threaded regions 413, 414 are positioned. The second threaded region 414 can define a longer length along the body 402 than each of the first threaded region 413 and the transition region 452. The first threaded region 413 can define a length that is the same as or different than a length defined by the transition region 452.

Depending on the intended application of the screw 400, the threading 412 at the first and second threaded regions can be the same threading or different threading. For example, the first threaded region 413 can include different threading 412 than the second threaded region 414. As one specific such example, threading 412 at the first threaded region 413 can have a different threading pitch or spacing between adjacent ridges than that defined by threading 412 at the second threaded region 414. As shown for the illustrated embodiment here, threading pitch or spacing between adjacent ridges defined by the threading 412 at the first threaded region 413 can be smaller than threading pitch or spacing between adjacent ridges defined by the threading 412 at the second threaded region 414. For instance, threading 412 defined at the first threaded region 413 can define a threading pitch or spacing that configures the first threaded region 413 as non-compressing threading, while threading 412 defined at the second threaded region 414 can define a threading pitch or spacing that configures the first threaded region 413 as a compressing threading that acts to move the bone portion at which the second threaded region 414 is positioned as the screw 400 is rotationally driven.

The transition region 452 can define at least one cutting feature 453. The transition region 452 can define the at least one cutting feature 453 in addition to or alternative to the transition region lacking threading 412. The at least one cutting feature 453 of the illustrated embodiment of the screw 400 includes a plurality of cutting flutes 454. The plurality of cutting flutes 454 can be circumferentially spaced about the central longitudinal axis 408 and spaced about a perimeter of the transition region 452. Each cutting flute of the plurality of cutting flutes 454 can extend out from the body 402 to define a cutting edge 455 as an outermost surface of each cutting flute 454 away from the body 402. The plurality of cutting flutes 454 can be configured to cut bone as the screw 400 is rotationally driven. The presence of the plurality of cutting flutes 454 can act to cut bone as the screw 400 is rotationally driven and thereby act to cause a reduction in stress force present at the bone portion where the plurality of cutting flutes 454 have cut bone. As such, the plurality of cutting flutes 454 can act to reduce stress in a bone portion that is to subsequently receive the first threaded region 413. This can be useful, as one example, where the body 402 of the screw 400 defines a non-uniform external screw body diameter.

As shown for the illustrated embodiment, an external screw body diameter 460 of the body 402 can be different at the first threaded region 413 than at the second threaded region 414. In particular, for this embodiment, the first threaded region 413 defines the external screw body diameter 460 as larger than the external screw body diameter 460 defined by the second threaded region 414, and the transition region 452 can define the external screw body diameter 460 that is different than at least one of the first and second threaded regions 413, 414.

The transition region 452 defining the at least one cutting feature 453 can facilitate self-countersinking for screw 400 during insertion. For example, the plurality of cutting flutes 454 can function to enlarge the opening in the bone into which first threaded region 413 is configured to be inserted. This can form an enlarged diameter opening in a region of bone proximal to where the second threaded region 414 is inserted, with the enlarged diameter opening being sized to receive first threaded region 413. As a result, screw 400 can be inserted to a depth where the end of first threaded region 413 is flush with or recessed relative to (countersunk) the external face of the bone.

At the proximal end 404 of the body 402, the screw 400 can include a driver receiving cavity 420. The driver receiving cavity 420 can define a sidewall 421, for instance a circumferential sidewall 421, along a length L of the at least a portion of the proximal end 404. The driver receiving cavity 420 can be configured to receive one or more types of drivers thereat to facilitate rotational driving the screw 400 via the driver received at the driver receiving cavity 420. For example, as will be described further herein, the driver receiving cavity 420 can be configured to receive both a screw-specific driver that corresponds to the screw 400 and a hexalobular driver. The hexalobular driver can have equally spaced lobes (e.g., six equally spaced lobes), whereas the screw-specific driver that corresponds to the screw 400 can have different spacing between lobes. As one such example, the screw-specific driver can have different spacing between each of its plurality of lobes, for instance such that the spacing between each of a plurality of lobes at the screw-specific driver corresponds, respectively, to each of a different sized groove (and thus to each of a different sized lobe) included at the driver receiving cavity 420 of the screw 400.

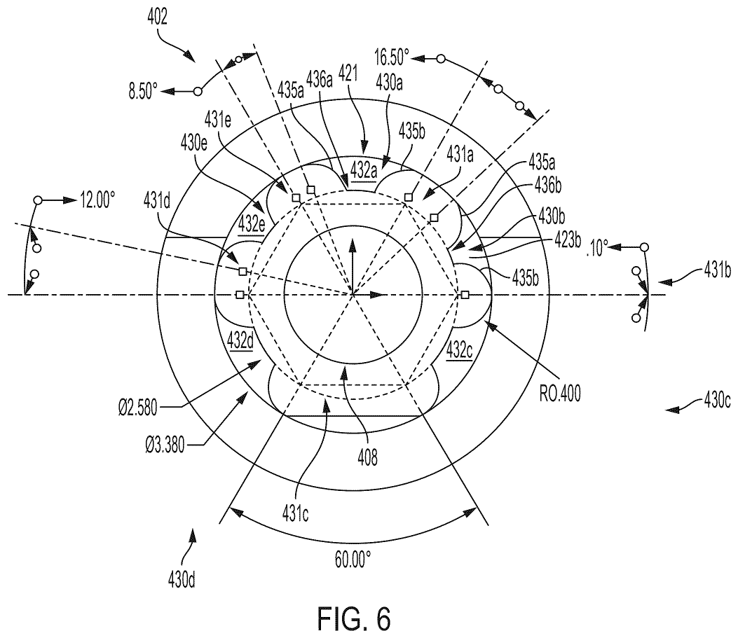

As noted, the driver receiving cavity 420 can include a plurality (e.g., at least three, at last four, at least five, at least six) of lobes 430 (seen, e.g., at FIG. 4B). The plurality of lobes 430 at the driver receiving cavity 420 can be circumferentially spaced apart from one another about the central longitudinal axis 408. The plurality of lobes 430 can define a plurality of grooves 431 (seen, e.g., at FIG. 4B). For example, each of the plurality of grooves 431 can be defined between two lobes 430 such that the plurality of grooves 431 alternate with the plurality of lobes 430 circumferentially around the driver receiving cavity 420 about the central longitudinal axis 408. As will be described further herein in reference to other drawing figures (e.g., FIGS. 6-9B), each of the plurality of lobes 430 can be of a different size, for instance, such that each of the plurality of lobes 430 defines a different internal lobular area 432. In one embodiment, the driver receiving cavity 420 can include at least three lobes 430 circumferentially spaced from one another about the central longitudinal axis 408 to define at least three grooves 431, with each of the at least three lobes 430 defining a different internal lobular area 432. In another embodiment, the driver receiving cavity 420 can include at least four lobes 430 circumferentially spaced from one another about the central longitudinal axis 408 to define at least four grooves 431, with each of the at least four lobes 430 defining a different internal lobular area 432. In a yet another embodiment, the driver receiving cavity 420 can include at least five lobes 430 circumferentially spaced from one another about the central longitudinal axis 408 to define at least five grooves 431, with each of the at least five lobes 430 defining a different internal lobular area 432. And in yet another embodiment, the driver receiving cavity 420 can include at least six lobes 430 circumferentially spaced from one another about the central longitudinal axis 408 to define at least six grooves 431, with each of the at least six lobes 430 defining a different internal lobular area 432.

The screw 400 can additionally include an alignment feature 470. The alignment feature 470 can be disposed along at least a portion of the body 402 of the screw 400. The alignment feature 470 can be configured to align with a predetermined anatomical feature of a patient when the screw 400 is positioned in and/or through a bone portion, such as a predetermined anatomical feature defined at the foot of the patient associated with a minimally invasive osteosynthesis (e.g., an outer surface of a metatarsal bone portion of the foot). For example, the body 402 can define the alignment feature 470 at the proximal end 404. This can include the alignment feature 470 being defined at the body 402 to form at least a portion of the proximal end 404. The illustrated embodiment shows the alignment feature 470 includes a bevel extending along at least a portion of the length L of the body 402 at the proximal end 404 (e.g., the alignment feature includes a bevel extending along a portion of the length of the first threaded region 413). The bevel of the alignment feature 470 can, in the exemplary application of an osteosynthesis at one or more bone portions of the foot, be configured to provide an alignment surface 471 at the alignment feature 470 that is configured to align with an outer surface of a bone portion (e.g., metatarsal portion) in the foot through which the screw 400 is positioned.

The plurality of differently sized lobes 430 at the screw 400 can be indexed relative to an alignment feature 470 also included at the screw 400. One example of this indexing of the plurality of differently sized lobes 430 relative to an alignment feature 470 at the screw 400 can be seen at FIG. 4B. An indexed positioning of the plurality of differently sized lobes 430 relative to the alignment feature 470 can help to configure the screw 400 for implantation positional driving relative to the bone portion(s) that is tailored to the particular desired, end goal implanted anatomic positioning of the screw relative to the one or more bone portion(s) that corresponds to the alignment feature 470 at the screw 400. As one example, the alignment feature 470 can include a bevel defined by the alignment surface 471, and this beveled alignment surface 471 can be configured to correspond to an outer surface of a bone portion, such as an outer surface of a metatarsal. Thus, when the body 402 defines a circular cross-sectional shape as for the illustrated embodiment of the screw 400, the bevel can extend along at least a portion of a length of that circular cross-sectional shaped body 402. The illustrated embodiment shows that the at least five differently sized lobes 430 are indexed relative to the location of the alignment feature 470 at the proximal end 404 of the body 402. Accordingly, engaging the screw 400 with a driver at these indexed lobes 430 can help to ensure positional accuracy of the screw's implantation orientation relative to one or more bone portions at which the screw is rotationally driven.

The plurality of differently sized lobes 430 at the screw 400 can be configured to engage different drivers for rotationally driving the screw 400. As noted previously, the driver receiving cavity 420 at the screw 400 can be configured to receive such different drivers for rotationally driving the screw 400. As such, the driver receiving cavity 420 can include a driver receptacle 422 that can include the plurality of differently sized lobes 430, and the driver receptacle 422, via the plurality of differently sized lobes 430, can be configured to receive both a screw-specific driver that corresponds to the screw 400 and a hexalobular driver to rotationally drive the screw 400. The driver receptacle 422 can extend from the proximal end 404 of the body 402 toward the distal end 406 of the body 402. The plurality of differently sized lobes 430 (e.g., at least three differently sized lobes 430; at least five differently sized lobes 430) can be included at the driver receptacle 422 spaced distally from the proximal end 404. Each of the plurality of differently sized lobes 430 (e.g., at least five differently sized lobes) can include at least two lobular sidewalls 435 that protrude radially inwardly into the driver receptacle 422. For example, each of the plurality of differently sized lobes 430 can be defined at least by a first lobular sidewall 435a and second lobular sidewall 435b that each protrude radially inwardly into the driver receptacle 422. And these first and second lobular sidewalls 435a, 435b can define, at least in part, the internal lobular area 432 of each of the lobes 430.

The screw 400 can additionally include a driver nub receiving bore 424. The driver nub receiving bore 424 can be defined at the body 402. The driver nub receiving bore 424 can extend a length along the central longitudinal axis 408. The driver nub receiving bore 424 can be spaced distally from the proximal end 404 and distally from the plurality of differently sized lobes 430. The driver nub receiving bore 424 can have a different internal body diameter than that at the axial location along the body 402 where the plurality of differently sized lobes 430 are located. For example, the driver nub receiving bore 424 can define a first internal body diameter 425 that is transverse to the central longitudinal axis 408, and each of the plurality of differently sized lobes 430 can define a second internal body diameter 426 that is transverse to the central longitudinal axis 408. For instance, the first internal body diameter 425, associated with the driver nub receiving bore 424, can be smaller than the second internal body diameter 426, associated with the axial location of the plurality of differently sized lobes 430 along the central longitudinal axis 408. Yet the first internal body diameter 425, associated with the driver nub receiving bore 424 can be large enough to receive therethrough a pin or wire, such as a K-wire, for instance a 1.4 mm K-wire of a 1.6 mm K-wire.

In use, when a driver is moved into engagement with the screw 400 at the proximal end 404, the driver can be received at the driver receiving cavity 420. As the driver is first received at the driver receiving cavity 420, the driver can first intersect the larger diameter of the second internal body diameter 426, associated with the axial location of the plurality of differently sized lobes 430, and then the driver can subsequently, upon additional axial movement of the driver relative to the screw, intersect the driver nub receiving bore 424.

FIG. 5 is a perspective view of an embodiment of a screw-specific driver 500. The screw-specific driver 500 can be configured to engage with the screw 400 to drive (e.g., rotationally drive) the screw 400 into one or more bone portions and/or to remove (e.g., rotationally remove) the screw 400 from one or more bone portions.

The screw-specific driver 500 can include a driver body 502. The driver body 502 can include a proximal end 504 and a distal end 506, and the driver body 502 can define a driver central longitudinal axis 508. The driver body 502 can extend a driver body length along the driver central longitudinal axis 508 from the proximal end 504 to the distal end 506. The distal end 506 of the driver body 502 can include a handle 507, and the handle 507 can be configured to be gripped by a hand of a user to impart a driving force at the screw-specific driver, for instance to impart a rotational driving force, to drive the screw 400 into one or more bone portions and/or to remove the screw 400 from one or more bone portions.

To help engage the screw 400 (e.g., and to help transfer driving force, such a torque, from the screw-specific driver 500 to the screw 400), the driver body 502 can also include a plurality of driving lobes 530 at the driver body 502. As shown for the illustrated example, the plurality of driving lobes 530 can be circumferentially spaced apart from one another about the driver central longitudinal axis 508. In one embodiment, the driver body 502 can include at least three driving lobes 530 at the driver body 502. In another embodiment, the driver body 502 can include at least five driving lobes 530 at the driver body 502. The number of driving lobes 530 included at the driver body 502 can be equal to the number of lobes 430 and/or number of grooves 431 at the screw 400. For example, the illustrated embodiment of the screw 400 includes five lobes 430 and five grooves 431, and, thus, the driver body 502 can include five driving lobes 530.

Each one of the plurality of driving lobes 530 can define a different driving lobular area 532. For example, the illustrated embodiment of the driver body 502 includes five driving lobes 530, and each of these five driving lobes 530 can define a different driving lobular area 532 than each of the other driving lobes 530 such that the driving lobular area 532 differs moving around the outer perimeter of the driver body 502 at the location of the driving lobes 530. Each driving lobe 530 can be defined by one or more driving lobular sidewalls 535 that extend out from the driver body 502, and such one or more driving lobular sidewalls 535 can form an outermost projection at or near the distal end 506. For the illustrated embodiment, each of the driving lobes 530 is formed by: (i) opposite driving lobular sidewalls 535 that merge together to form an outermost projection at each driving lobe 530 and (ii) a nub sidewall 537 that forms an innermost surface at each driving lobe 530 and is the point at which each driving lobe 530 extends out from the driver body 502. Thus, by using varied the lobular sidewalls 535 to define each driving lobe 530, each driving lobe 530 can define a different driving lobular area 532.

Thus, just as each of the lobes 430 at the screw 400 can define a different internal lobular area 432, each of the driving lobes 530 can be of a different internal lobular area 532. As noted elsewhere herein, each of the lobes 430 can in turn define adjacent grooves 431 therebetween and, because each of the lobes 430 can have a different internal lobular area, each of the grooves 431 at the screw 400 can be of a different area (e.g., different internal groove area). The driving lobes 530 can be configured to engage the grooves 431 defined at the screw 400 to thereby drive the screw 400. As such, each of the driving lobes 530 at the screw-specific driver 500 can be of a different internal lobular area 532, and the different internal lobular area 532 defined by each driving lobe 530 can correspond, respectively, to each of the different areas defined by each of the grooves 431. As one example where the screw-specific driver 500 includes at least three driving lobes 530 that each define a different driving lobular area 532, each one of these at least three different driving lobular areas 532 can correspond, respectively, to one of at least three grooves 431 at the screw 400, each of which defines a different internal groove area. As another example, where the screw-specific driver 500 includes at least five driving lobes 530 that each define a different driving lobular area 532, each one of these at least five different driving lobular areas 532 can correspond, respectively, to one of at least five grooves 431 at the screw 400, each of which defines a different internal groove area.

The screw-specific driver 500 can also include a driver engagement nub 524. The driver engagement nub 524 can be at the distal end 506 of the driver body 502. The driver engagement nub 524 can be configured to engage the screw 400, such as within the driver receiving cavity 420, for instance, at the driver nub receiving bore 424 at the screw 400. As shown for the illustrated embodiment, the plurality of driving lobes 530 can be spaced proximally from the driver engagement nub 524. For instance, the plurality of driving lobes 530 can begin at a location along the driver body 502 that is proximally set back along the driver body 502 at least from a proximal end of the driver engagement nub 524. With this positioning of the plurality of driving lobes 530 relative to the driver engagement nub 524, the driver engagement nub 524 can be configured to engage the screw 400 prior to the plurality of driving lobes 530 engaging the screw 400.

The relative positioning of the driver engagement nub 524 and plurality of driving lobes 530 at the screw-specific driver 500 with respect to the relative positioning of the driver nub receiving bore 424 and the plurality of lobes 430 and grooves 431 at the screw 400 can provide a driver engagement alignment function. For example, because the plurality of driving lobes 530 can be proximally offset from the driver engagement nub 524 at the screw-specific driver 500 and the driver nub receiving bore 424 can be distally offset from the plurality of lobes 430 and grooves 431, as the distal end 506 of the screw-specific driver 500 is inserted within the driver receiving cavity 420 at the screw 400, the driver engagement nub 524 can pass the plurality of lobes 430 and grooves 431 to enter and sit at the driver nub receiving bore 424 at the screw 400. This can result in the trailing driving lobes 530 being positioned to interface with the lobes 430 and grooves 431 and, in some embodiments, placing the driving lobes 530 at the grooves 431 can be caused by imparting relative rotation between the screw-specific driver 500 and the screw 400 until the driving lobes 530 are rotationally aligned with the grooves 431 such that driving lobes 530 can then axially slide into the grooves 431 with continued further axial insertion of the screw-specific driver 500 into the driver receiving cavity 420. Accordingly, the driver engagement nub 524 can provide a first alignment function at the driver receiving cavity 420 (e.g., axial alignment with the driver nub receiving bore 424) and once this axial alignment is present rotational alignment between the driving lobes 530 and the grooves 431 can be imparted to then allow further axial insertion of the screw-specific driver 500 into the driver receiving cavity 420 to cause the driving lobes 530 to sit within the grooves 431.

To help facilitate this alignment feature, the driver engagement nub 524 can be sized to correspond to a size of the driver nub receiving bore 424 such that the driver engagement nub 524 can be slid within the driver nub receiving bore 424, but the driving lobes 530 can be sized to prevent insertion of the driving lobes 530 within the driver nub receiving bore 424. As one example, the driver engagement nub 524 can define an external diameter at the driver body 502 that is less than an external diameter at the driver body 502 associated with the plurality of driving lobes 530. For instance, the driver engagement nub 524 can define a first external driver body diameter 560 that is transverse to the driver central longitudinal axis 508, and each of the plurality of driving lobes 530 can define a second external driver body diameter 561 that is transverse to the driver central longitudinal axis 508. The first external driver body diameter 560, at the driver engagement nub 524, can be smaller than the second external driver body diameter 561, at the plurality of driving lobes 530.

FIG. 6 is a plan view of one embodiment of a plurality of lobes 430 that can be included at the screw 400. FIG. 6 includes illustrative dimensions for illustrative purposes and other dimensions can be used within the scope of the present disclosure.

The illustrated embodiment shows that the screw 400 can include at least five lobes 430a-430e circumferentially spaced from one another about the central longitudinal axis 408 to define at least five grooves 431a-431e also circumferentially spaced from one another about the central longitudinal axis 408. The lobes 430a-430e and grooves 431a-431e can alternate with one another moving circumferentially around the central longitudinal axis 408 at the driver receiving cavity. Each of these at least five lobes 430a-430e can define a different internal lobular area 432a-432e, respectively. Though the features and aspects disclosed herein in reference to the lobes 430 and grooves 431 as shown at the example of FIG. 6 can be applied to other embodiments within the scope of this disclosure having different numbers of lobes 430 and grooves 431, such as at least three lobes 430 circumferentially spaced from one another about the central longitudinal axis 408 to define at least three grooves 431, with each of the at least three lobes 430 defining a different internal lobular area 432. For example, for one screw 400 embodiment, such as that illustrated, the at least five lobes 430a-430e can be only five lobes 430a-430e and the at least five grooves 431a-431e can be only five grooves 431-431e, with each of five grooves 431a-431e defining a different area than each other of the other of the five grooves 431a-431e.

As seen at FIG. 6, the driver receiving cavity 420 can define the sidewall 421. Each of the plurality of lobes 430a-430e can extend out from the sidewall 421 to an innermost projecting surface 436a. For example, each of the plurality of lobes 430a-430e can extend out from the sidewall 421 to an innermost projecting surface 436a at a location spaced from and axially between the proximal end of the body 402 and the driver nub receiving bore. Each of the plurality of lobes 430a-430e can define an internal lobular area 432a-432e, respectively. The internal lobular area 432a-432e defined by each lobe 430a-430e can be defined by a region extending from the sidewall 421 to the innermost projecting surface 436a. For instance, each lobe 430a-430e can be defined by the sidewall 421 and at least two lobular sidewalls 435a, 435b that extend out from the sidewall 421 and merger together to form the innermost projecting surface 436a at each of the plurality of lobes 430a-430e. And each groove 431a-431e can be defined between two adjacent lobes 430a-430e. For instance, groove 431a can be defined between lobes 430a and lobe 430b. This can include groove 431a being defined by the sidewall 421 (e.g., the same sidewall that defines, at least in part, the lobes 430a-430e) as well as the sidewall 435b associated with the lobe 430a and the sidewall 435a associated with the lobe 430b.

As noted elsewhere herein, the internal lobular area 432a-432e can differ for each of the lobes 430a-430. As one example, the internal lobular area 432a-432e can differ for each of the lobes 430a-430 by using different spacing between sidewalls 435a, 435b that form each lobe 430. This in turn can also cause the area defined at each groove 431a-431e to differ from each other too. The ratio or magnitude of the difference in area between each of the internal lobular areas 432a-432e can differ depending on the embodiment. Likewise, the ratio or magnitude of the difference in area between each of the internal groove areas defined by each of the grooves 431a-431e can differ depending on the embodiment. For instance, the internal lobular area 432 of one of the at least five lobes 430a-430e can be at least twice as large as the internal lobular area 432 of at least one other of the at least five lobes 430a-430e, and the internal groove area of one of the at least five grooves 431a-431e can be at least twice as large as the internal groove area of at least one other of the at least five grooves 431a-431e. For instance, referring to the example lobes 430a-430e and grooves 431a-431e shown at FIG. 6, the internal lobular area 432d of the lobe 430d can be at least twice as large as the internal lobular area 432b of the lobe 430b and the internal groove area of the groove 431c can be at least twice as large as the internal groove area of the groove 431b.

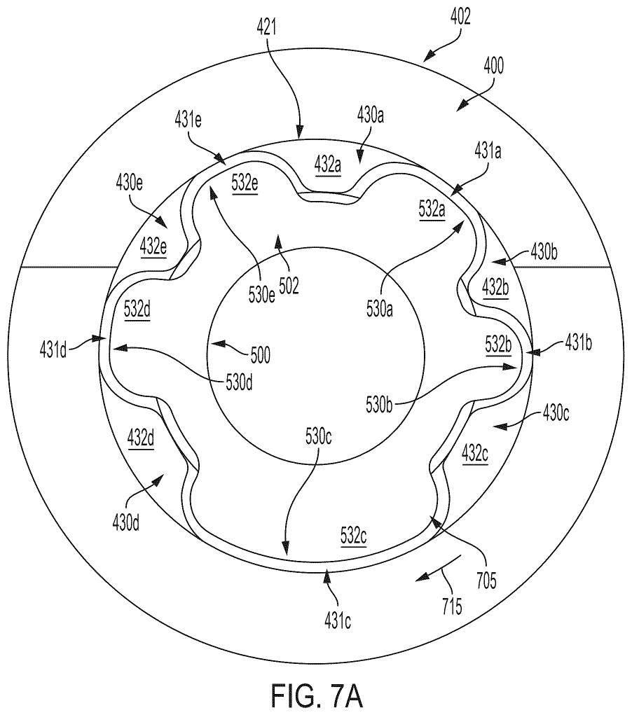

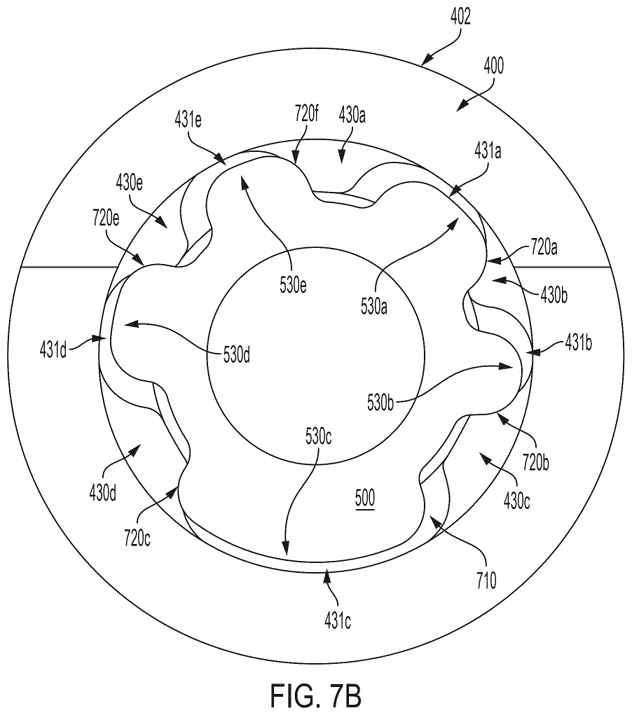

FIGS. 7A and 7B illustrate each of the plurality of lobes 430a-430e and grooves 431a-431e at the screw 400 relative to driving lobes 530a-530e at screw-specific driver 500 when the screw-specific driver 500 is engaged at the screw 400 (e.g., engaged at the driver receiving cavity). FIG. 7A is a plan view of the driving lobes 530a-530e relative to the plurality of lobes 430a-430e of the screw 400 when the screw-specific driver 500 is at a first screw-specific driver rotational position 705, and FIG. 7B is a plan view of the driving lobes 530a-530e relative to the plurality of lobes 430a-430e of the screw 400 when the screw-specific driver 500 is at a second, different screw-specific driver rotational position 710. The second screw-specific driver rotational position 710 can be arrived at from the first screw-specific driver rotational position 704 by rotationally driving the screw-specific driver 500 in the rotational direction 715 relative to the screw 400. For example, the rotational direction 715 can correspond to driving the screw 400 into one or more bone portions, and an opposite rotational direction of the rotational direction 715 can correspond to removing the screw 400 from one or more bone portions.

When the screw-specific driver 500 is at the first screw-specific driver rotational position 705, driving lobes 530a-530e can be positioned within grooves 431a-431e (e.g., the driving lobe internal area 532a-532e can be less than the groove internal area to facilitate placement within each of the groove internal areas). Then, as the screw-specific driver 500 is rotationally driven in the direction 715, one or more of the plurality of driving lobes 530a-530e is configured to engage an adjacent driving lobe 530a-530e of the screw-specific driver 500. For example, as the screw-specific driver 500 is rotationally driven in the direction 715 from the first screw-specific driver rotational position 705 to the second screw-specific driver rotational position 710, one or more of the plurality of driving lobes 530a-530e can be brought into contact with one or more of the lobes 430a-430e at the screw 400. As one more specific example, as the screw-specific driver 500 is rotationally driven in the direction 715 from the first screw-specific driver rotational position 705 to the second screw-specific driver rotational position 710, two or more of the plurality of driving lobes 530a-530e can be brought into contact with two or more of the lobes 430a-430e at the screw 400.

The illustrated embodiment includes internal lobular areas 432 and related grooves 431 and corresponding, complementary internal driving lobular areas 532 such that as the screw-specific driver 500 is rotationally driven in the direction 715 from the first screw-specific driver rotational position 705 to the second screw-specific driver rotational position 710, each of the five driving lobes 530a-530e is brought into contact with each of the lobes 430a-430e at the screw 400. FIG. 7B shows this with contact point 720a between driving lobe 530a and lobe 430b (e.g., contact at sidewall 435a), contact point 720b between driving lobe 530b and lobe 430c, contact point 720c between driving lobe 530c and lobe 430d, contact point 720d between driving lobe 530d and lobe 430e, contact point 720e between driving lobe 530e and lobe 430f, and contact point 720f between driving lobe 530f and lobe 430a. Likewise, the illustrated embodiment can further include the internal lobular areas 432 and related grooves 431 and corresponding, complementary internal driving lobular areas 532 such that as the screw-specific driver 500 is rotationally driven in the direction opposite the direction 715 from the second screw-specific driver rotational position 710 to a third screw-specific driver rotational position in the direction opposite the direction 715, each of the five driving lobes 530a-530e can be brought into contact with each of the lobes 430a-430e at the screw 400.

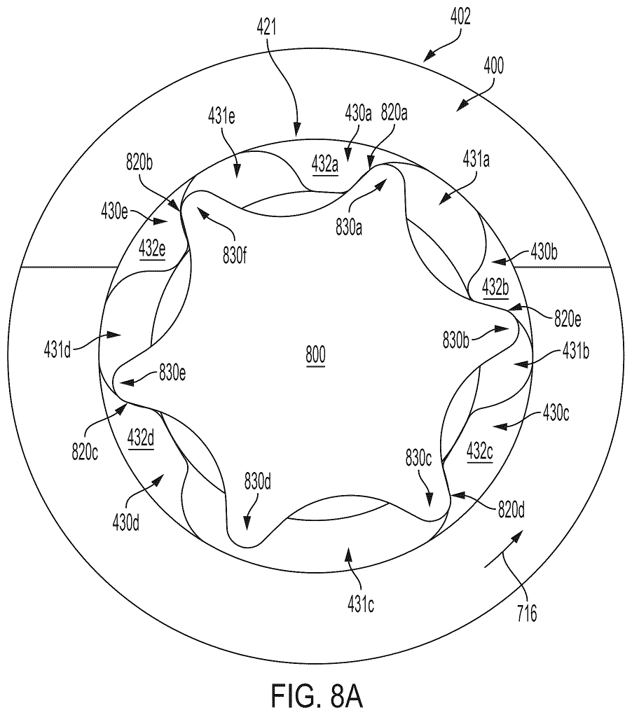

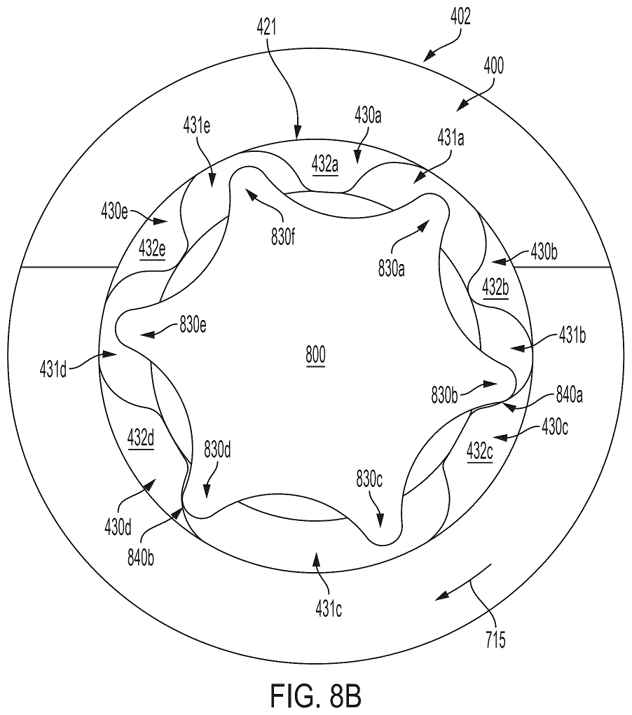

FIGS. 8A and 8B illustrate the plurality of lobes 430a-430e and grooves 431-431e at the screw 400 relative to driving lobes 830 at an embodiment of a hexalobular driver 800 when the hexalobular driver 800 is engaged at the screw 400. As noted elsewhere herein, the screw 400 can be configured to receive and engage the screw-specific driver 500 as well as a hexalobular driver 800. The hexalobular driver 800 can include six driving lobes 830a-830f, and the hexalobular driver 800 can include an equal spacing between each pair of driving lobes 830a-830f moving circumferentially around a screw engagement end of the hexalobular driver 800. FIG. 8A is a plan view of lobes 430a-430e engaging driving lobes 830a-830f at hexalobular driver 800 in a first hexalobular driver rotational direction 716, and FIG. 8B is a plan view of lobes 430a-430e engaging driving lobes 830a-830f at hexalobular driver 800 in a second, opposite hexalobular driver rotational direction 715.

As shown at FIGS. 8A, one or more of the plurality of lobes 430a-430e at the screw 400 is configured to engage the hexalobular driver 800 in the first hexalobular driver rotational direction 716, which can correspond to removal of the screw 400. For example, at least two of the plurality of lobes 430a-430e can be configured to engage the hexalobular driver 800 in the first hexalobular driver rotational direction 716. The illustrated example at FIG. 8A shows that each of the five lobes 430a-430e can be configured to contact one of the driving lobes 830a-830f such that five lobes 430a-430e contact five of the six driving lobes 830a-830f. In particular, lobe 430a is configured to contact driving lobe 830a at contact point 820a, lobe 430e is configured to contact driving lobe 830f at contact point 820b, lobe 430d is configured to contact driving lobe 830e at contact point 820c, lobe 430c is configured to contact driving lobe 830c at contact point 820d, and lobe 430b is configured to contact driving lobe 830b at contact point 820e when hexalobular driver 800 is engaged at screw 400 and driven in the first hexalobular driver rotational direction 716. The illustrated example at FIG. 8A also shows that one of the six driving lobes 830d is configured to not contact a lobe at the screw 400 when hexalobular driver 800 is engaged at screw 400 and driven in the first hexalobular driver rotational direction 716. As such, when hexalobular driver 800 is engaged at screw 400 and driven in the first hexalobular driver rotational direction 716, one groove 431c can be configured to receive two of the driving lobes 830c, 830d at that same, one groove 431c while each of the other grooves 431a, 431b, 431d, 431e can be configured to receive one driving lobe 830a, 830b, 830e, 830f respectively.

As shown at FIG. 8B, one or more of the plurality of lobes 430a-430e at the screw 400 is configured to engage the hexalobular driver 800 in the second hexalobular driver rotational direction 715, which can correspond to advancement of the screw 400. The second hexalobular driver rotational direction 715 can be opposite the first hexalobular driver rotational direction 716. For example, at least two of the plurality of lobes 430a-430e can be configured to engage the hexalobular driver 800 in the second hexalobular driver rotational direction 715. The illustrated example at FIG. 8B shows that only two of the five lobes 430a-430e can be configured to contact driving lobes 830a-830f such that only two lobes contact two of the six driving lobes 830a-830f. In particular, lobe 430c is configured to contact driving lobe 830b at contact point 840a and lobe 430d is configured to contact driving lobe 830d at contact point 840b when hexalobular driver 800 is engaged at screw 400 and driven in the second hexalobular driver rotational direction 715. One or more of the six driving lobes 830a-830f can be configured to not contact a lobe at the screw 400 when hexalobular driver 800 is engaged at screw 400 and driven in the second hexalobular driver rotational direction 715. The illustrated example at FIG. 8B also shows that the lobes 430a-430e and grooves 431a-431e are configured such that four driving lobes 830a, 830c, 830e, 830f do not contact a lobe at the screw 400 when hexalobular driver 800 is engaged at screw 400 and driven in the second hexalobular driver rotational direction 715. Thus, the lobes 430a-430e and grooves 431a-431e can be configured such that a different number of the plurality of lobes 430a-430e is configured to engage the hexalobular driver 800 in the first hexalobular driver rotational direction 716 than in the second hexalobular driver rotational direction 715. Moreover, the configuration of the lobes 430a-430e and grooves 431a-431e can result in two driving lobes 830c, 830d received at the single groove 431c, for instance, when hexalobular driver 800 is engaged at screw 400 and driven in both the first and second hexalobular driver rotational directions 716, 715.

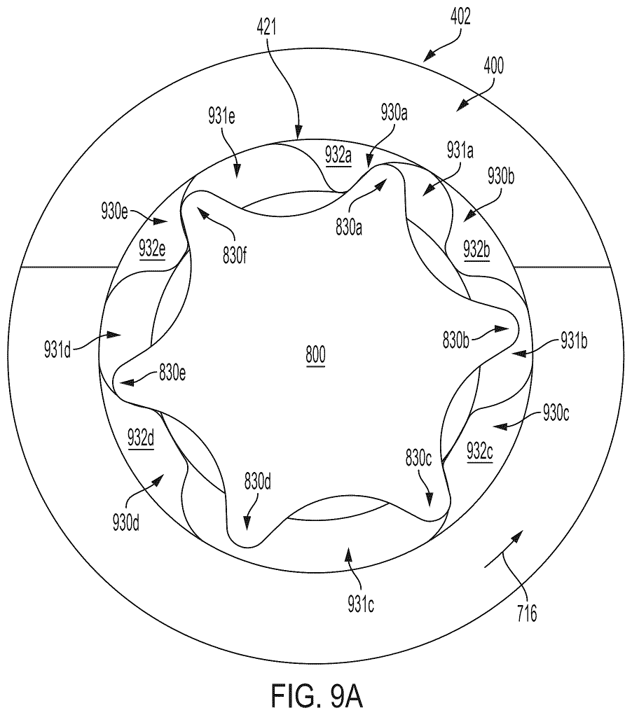

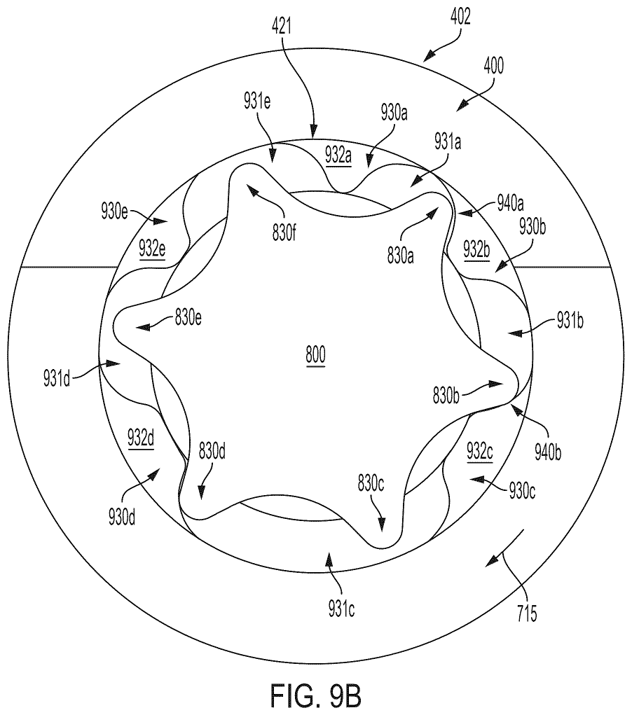

FIGS. 9A and 9B illustrate another embodiment of a plurality of lobes 930a-930e that can be included at the screw 400, for instance in place of the plurality of lobes 430a-430e. The plurality of lobes 930a-930e shown for the embodiment at FIGS. 9A and 9B can be the same as the plurality of lobes 430a-430 as disclosed elsewhere herein except that the plurality of lobes 930a-930e of the embodiment at FIGS. 9A-9B are sized differently than the plurality of lobes 430a-430e of the embodiment at FIGS. 8A-8B. Likewise, the plurality of grooves 931a-931e shown for the embodiment at FIGS. 9A and 9B can be the same as the plurality of grooves 431a-431e as disclosed elsewhere herein except that the plurality of grooves 931a-931e of the embodiment at FIGS. 9A-9B are sized differently than the plurality of grooves 431a-431e of the embodiment at FIGS. 8A-8B. Each of the plurality of lobes 930-930 can define a different, respective internal lobular area 932a-932e than each of the other of the plurality of lobes 930a-930e, and thus, each of the grooves 931a-931c can define a different, respective internal groove area than each of the other of the plurality of grooves 931a-931e.

FIG. 9A is a plan view of lobes 930a-930e engaging driving lobes 830a-830f at hexalobular driver 800 in the first hexalobular driver rotational direction 716, and FIG. 9B is a plan view of lobes 930a-930e engaging driving lobes 830a-830f at hexalobular driver 800 in the second, opposite hexalobular driver rotational direction 715. Comparing the position of the driving lobes 830a-830f at hexalobular driver 800 relative to the lobes 930a-930e and grooves 931a-931e at the second hexalobular driver rotational direction 715 of FIG. 9B to that at the second hexalobular driver rotational direction 716 of FIG. 8B shows that changing the internal lobular areas (internal lobular areas 432a-432e versus different internal lobular areas 932a-932e) can change engagement between the driving lobes 830a-830f and the screws' lobes 930a-930e. Namely, at FIG. 9B, when the hexalobular driver 800 is at the second hexalobular driver rotational direction 715, lobe 930b is configured to contact driving lobe 830a at contact point 940a, whereas at the embodiment of FIG. 8B when the hexalobular driver 800 is at the second hexalobular driver rotational direction 715 lobe 430b is configured to be spaced further from driving lobe 830a than is the case for lobe 930b at the embodiment of FIG. 9B.

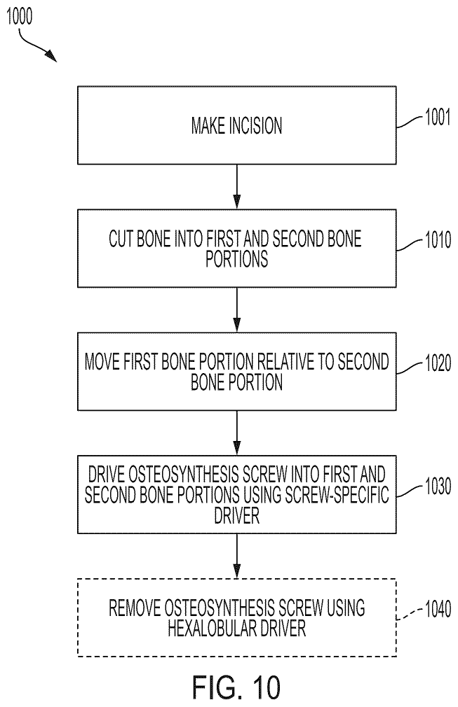

FIG. 10 is a flow diagram of an embodiment of a bone fixation technique 1000. The example technique 1000 will be described with respect to first metatarsal 210, although can be performed on other bones, as discussed above. The technique 1000 can use any one or more of the features disclosed elsewhere herein, including any one or more features disclosed herein with respect to the screw 400, screw-specific driver 500, and hexalobular drive 800.

At step 1001, the technique 1000 includes making an incision through a skin of a patient and retracting the skin along the incision to expose one or both of the first metatarsal bone portion and the second metatarsal bone portion. For example, to surgically access the bone, the patient may be placed in a supine position on the operating room table and general anesthesia or monitored anesthesia care administered. Hemostasis can be obtained by applying thigh tourniquet or mid-calf tourniquet prior to making the incision. In some examples, the clinician may additionally image at least a portion of the foot 200 where first metatarsal 210 is to be cut and, correspondingly, the incision is to be made. The clinician may take a fluoroscopic images of at least a portion of foot 200 in one or more views encompassing the region where first metatarsal 210 is to be cut. The clinician can identify the midline of first metatarsal 210 (midline between the dorsal-most surface and the planter-most surface) on the medial side of the foot based on the imaging. The clinician may position a K-wire or other radiopaque instrument along the midline while viewing the foot under imaging to identify the midline location. The clinician can also identify the metaphysis region, e.g., the first MTP joint 232 between first metatarsal 210 and proximal phalanx 220. The clinician may position a K-wire or other radiopaque instrument at the MTP joint while viewing the foot under imaging to identify the MTP joint.

Before or after making an incision at a target location offset from MTP joint 232 by a distance, the clinician may insert a K-wire into first metatarsal 210. The K-wire can be inserted at the midline of first metatarsal 210 (midline in the dorsal-to-plantar direction) and can extend medially outwardly from a remainder of the foot. In either case, the clinician can make an incision substantially centered about the target cut location and/or K-wire positioned at the target cut location. The clinician can make the incision by guiding a cutting instrument (e.g., scalpel) along the location wherein skin is to be cut with or without the aid of an incision guide define a guide surface (e.g., cut slot) for controlling the length of the skin incision. In practice, the incision may have a length within a range from 5 mm to 30 mm, such as from 10 mm to 25 mm, or from 15 mm to 20 mm. The incision may extend distal-to-proximally along the length of the metatarsal and/or dorsal-to-plantarly about the circumferential perimeter of the metatarsal.

At step 1010, the technique 1000 includes cutting first metatarsal 210 into the first metatarsal bone portion and the second metatarsal bone portion. For example, with first metatarsal 210 exposed through the incision in the skin, the example technique 1000 can include cutting the first metatarsal 210 to form a distal metatarsal portion and a proximal metatarsal portion. For instance, after creating an incision through the skin at a target location where first metatarsal 210 is to be cut, the clinician may insert a cutting instrument through the incision through the first metatarsal. In some examples, the clinician may cut first metatarsal 210 freehand by controlling the positioning and movement of the cutting instrument with their hand without the aid of a guide. In other examples, the clinician may cut first metatarsal 210 with the aid of a cutting guide (which can also be referred to as a bone preparation guide) having a guide surface positionable over first metatarsal 210 at the location with the bone is to be cut. When using a bone preparation guide, a cutting instrument can be inserted against a guide surface (e.g., between a slot define between two guide surfaces) to guide the cutting instrument for bone cutting. Example cutting instruments that can be used to cut first metatarsal 210 (which may also be referred to as tissue removing instruments) include, but are not limited to, a saw blade, a rotary bur, a rongeur, a reamer, an osteotome, a curette, and the like. In some examples, the clinician may use one cutting instrument (e.g., saw blade, rotary bur) to transect first metatarsal 210 into two portions and then further prepared the cut end faces of the two bone portions, e.g., by fenestrating, morselizing, and/or otherwise generating bleeding bone faces to promote fusion.

At step 1020, the technique 1000 includes moving one of the first metatarsal bone portion and the second metatarsal bone portion relative to the other of the first metatarsal bone portion and the second metatarsal bone portion. This step 1020 can be taken before driving the osteosynthesis screw at step 1030. For example, step 1020 can include moving the cut distal metatarsal portion relative to the cut proximal metatarsal portion, for instance, to help correct an anatomical deformity. In one particular such example, the clinician can shift the distal metatarsal portion in the transverse plane (e.g., move the distal metatarsal portion laterally), rotate the distal metatarsal portion in the frontal plane, and/or shift the distal metatarsal portion in the sagittal plane. In some implementations, the clinician can engage the distal metatarsal portion with a bone positioning device operable to controllably move the distal metatarsal portion relative to the proximal metatarsal portion.

At step 1030, the technique 1000 includes driving an osteosynthesis screw (e.g., screw 400) into the first metatarsal bone portion and the second metatarsal bone portion using a screw-specific driver (e.g., screw-specific driver 500). The osteosynthesis screw can be rotationally driven into first metatarsal bone portion and the second metatarsal bone portion before, after, and/or while moving the distal metatarsal portion of the cut metatarsal relative to the proximal metatarsal portion in one or more planes. This can include rotationally driving the osteosynthesis screw attached to the screw-specific driver through the first metatarsal bone portion and into the second metatarsal bone portion and across a separation between the first and second metatarsal bone portions.

For example, step 1030 may include attaching the osteosynthesis screw to the screw-specific driver. The screw-specific driver can be engaged with a driver receiving cavity of the osteosynthesis screw. This driver receiving cavity can include at least three lobes circumferentially spaced apart from one another about a screw central longitudinal axis to define at least three grooves. Each one of the at least three lobes can define a different internal lobular area, and the at least three grooves can be configured to receive a hexalobular driver having equally spaced lobes that is different than the screw-specific driver having asymmetrically sized and/or spaced lobes. In some applications of the technique 1000, after rotationally driving the osteosynthesis screw, the screw-specific driver can be disengaged from the osteosynthesis screw by disengaging the at least three driving lobes of the screw-specific driver from the at least three grooves of the osteosynthesis screw.

The technique 1000 can include an optional step 1040 of removing the osteosynthesis screw using a hexalobular driver. For example, after rotationally driving the osteosynthesis screw and disengaging the screw-specific driver from the osteosynthesis screw, the hexalobular driver can be attached to the osteosynthesis screw and the osteosynthesis screw can be removed using the hexalobular driver. For instance, the hexalobular driver can be attached to the osteosynthesis screw and the osteosynthesis screw can be removed by rotationally driving the hexalobular driver coupled to the osteosynthesis screw. In some examples, the number of driving lobes at the hexalobular driver that contact screw lobes for removing the osteosynthesis screw can be different than the number of driving lobes at the screw-specific driver that contact screw lobes for rotationally driving the osteosynthesis screw into the bone portions at step 1030.

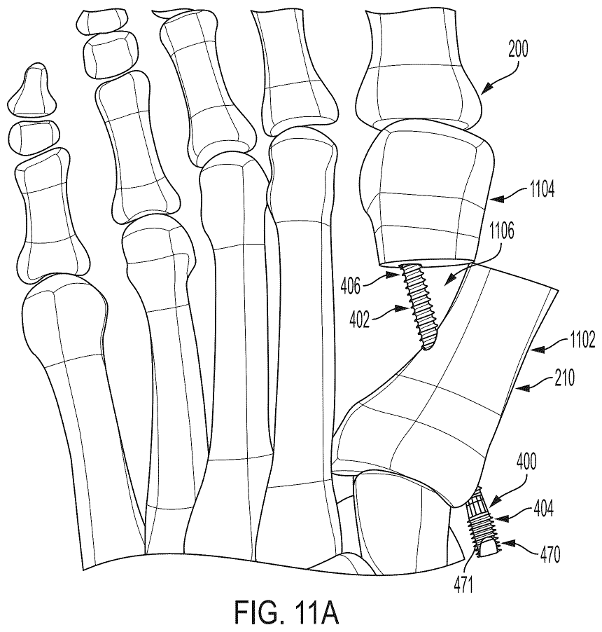

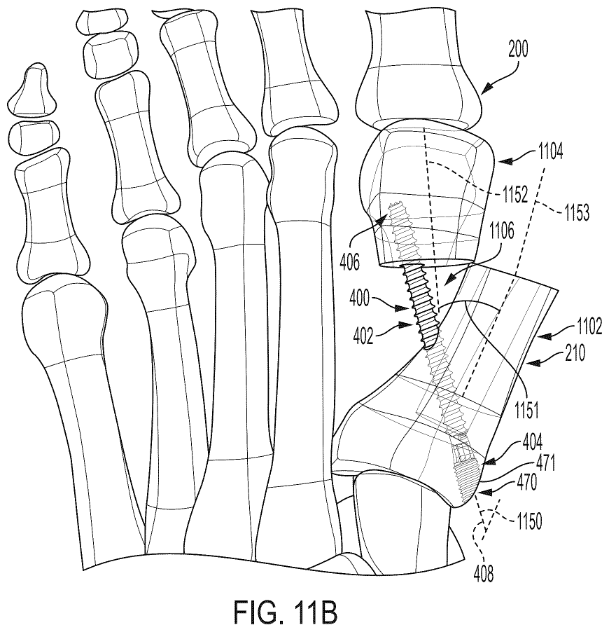

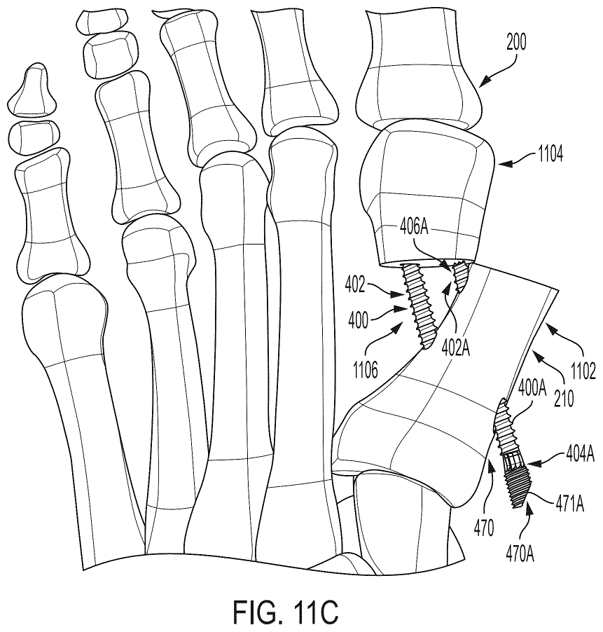

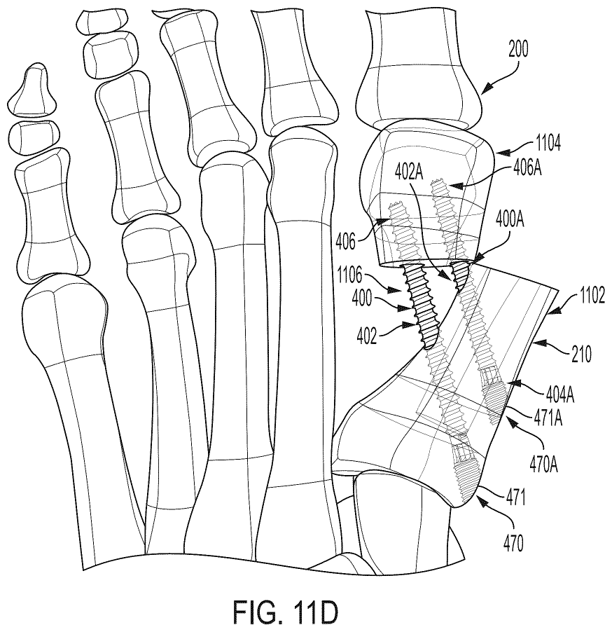

FIGS. 11A-11D illustrate an exemplary sequence of implanting, and resulting implanted orientation of, one or more screws through a first bone portion, into a second bone portion, and across a separation between the first and second bone portions. FIGS. 11A and 11B illustrate a sequence for implanting one screw through the first bone portion, into the second bone portion, and across the separation between the first and second bone portions. And FIGS. 11C and 11D illustrate a sequence for implanting a second screw through the first bone portion, into the second bone portion, and across the separation between the first and second bone portions. For instance, as shown at FIGS. 11C and 11D, a second screw can be implanted, spaced apart from the first screw after the first screw has been implanted, through the first bone portion, into the second bone portion, and across the separation between the first and second bone portions. The screw(s) shown at FIGS. 11A-11D can be similar to, or the same as, one or more screw embodiments disclosed elsewhere herein (e.g., at FIGS. 4A-4B). As such, in describing the examples shown at FIGS. 11A-11D, the illustrated screw(s) will be referred to as screw(s) 400 as disclosed for embodiments elsewhere herein. Though other embodiments associated with implanting a screw through, and orienting an implanted screws at, the first bone portion, into the second bone portion, and across the separation between the first and second bone portions can include use of other screw device embodiments.

FIGS. 11A and 11B show an exemplary sequence for implanting screw 400 through a first bone portion 1102, into a second bone portion 1104, and across a separation 1106 between the first and second bone portions 1102, 1104. FIG. 11A shows the screw 400 implanted through first bone portion 1102 with proximal end 404 of the screw 400 encountering first bone portion 1102, body 402 of screw 400 extending across the separation 1106, and distal end 406 of the screw 400 encountering the second portion 1104. The screw 400 can have distal end 406 initially placed at first bone portion 1102 and screw 400 driven (e.g., at proximal end 404) to advance screw 400 through first bone portion 1102 and to extend screw 400 across the separation 1106 such that the distal end 406 encounters the second portion 1106 as shown at FIG. 11A. Then, upon further driving of screw 400 as shown at FIG. 11A, distal end 406 of screw 400 can be advanced into second portion 1104 as shown at FIG. 11B.

As shown at FIG. 11B, screw 400 can be implanted at foot 200 such that distal end 406 of screw 400 is at (e.g., within) second bone portion 1104, proximal end 404 of screw 400 is at (e.g., at least partially within) first bone portion 1106, and a portion of body 402 of screw 400 is across the separation 1106 between the first and second bone portions 1102, 1104. As shown at FIG. 11B, proximal end 404 of screw 400 can be implanted at (e.g., at least partially within) first bone portion 1102 such that alignment feature 470, at screw 400, is aligned with a predetermined anatomical feature of foot 200 when distal end 406 is at (e.g., within) second bone portion 1104 and a portion of body 402 is across separation 1106. For example, as shown at FIG. 11B, the predetermined anatomical feature defined at the foot 200 can be associated with a minimally invasive osteosynthesis. As one such example shown at FIG. 11B, the predetermined anatomical feature defined at foot 200 and associated with a minimally invasive osteosynthesis can be an outer surface of first bone portion 1102 (e.g., as shown at FIG. 11B an outer surface of a metatarsal bone portion of the foot, such as an outer medial surface of a first metatarsal bone portion, for instance an outer medial surface of a first, proximal metatarsal bone portion). Thus, as shown at FIG. 11B, screw 400 can be implanted such that alignment feature 470 is aligned with the outer surface of first bone portion 1102 (e.g., as shown at FIG. 11B aligned with the outer medial surface of a first metatarsal bone portion) while distal end 406 is at (e.g., within) second bone portion 1104 and a portion of body 402 is across separation 1106. This can include, as shown at FIG. 11B, alignment surface 471 is aligned with the outer surface of first bone portion 1102 (e.g., as shown at FIG. 11B aligned with the outer medial surface of a first metatarsal bone portion) while distal end 406 is at (e.g., within) second bone portion 1104 (e.g., second portion of first metatarsal) and a portion of body 402 is across separation 1106. Such orientation of the implanted screw 400 using alignment feature 470 at bone portion 1102 can be useful in providing a visual indication of intended screw implantation orientation at foot 200 relative to one or more predetermined anatomical feature(s) defined at foot 200.

FIGS. 11C and 11D show screw 400 as implanted and described in reference to FIGS. 11A and 11B. FIGS. 11C and 11D additionally illustrate implantation of a second screw 400A through first bone portion 1102, into second bone portion 1104, and across separation 1106 between first and second bone portions 1102, 1104. As shown at FIGS. 11C and 11D, second screw 400A can be so implanted at first and second bone portions 1102, 1104 at a location that is spaced apart, about first and second bone portions 1102, 1104, from first screw 400. Second screw 400A can thus be implanted after first screw 400 has been implanted through first bone portion 1102, into second bone portion 1104, and across separation 1106 between first and second bone portions 1102, 1104. FIG. 11C shows second screw 400A implanted through first bone portion 1102 with body 402A of second screw 400A extending across separation 1106, and distal end 406A of second screw 400A encountering second portion 1104.

Then, upon further driving of second screw 400A as shown at FIG. 11C, distal end 406A of second screw 400A can be advanced into second portion 1104 as shown at FIG. 11D. As shown at FIG. 11D, first and second screws 400, 400A can be implanted at foot 200 such that respective distal ends 406, 406A of screws 400, 400A are each at (e.g., within) second bone portion 1104, respective proximal ends 404, 404A of screws 400, 400A are each at (e.g., at least partially within) first bone portion 1102, and portions of respective bodies 402, 402A of screws 400, 400A are across separation 1106 between the first and second bone portions 1102, 1104. In a more specific such example, shown at FIG. 11D, respective proximal ends 404, 404A of screws 400, 400A can be implanted such that respective alignment features 470, 470A are each aligned with the outer surface of first bone portion 1102 (e.g., as shown at FIG. 11D each aligned with the outer medial surface of a first metatarsal bone portion) while respective distal ends 406, 406A of screws 400, 400A are at (e.g., within) second bone portion 1104 and respective portions of body 402, 402A of screws 400, 400A are across separation 1106. This can include, as shown at FIG. 11D, respective alignment surfaces 471, 471A of screws 400, 400A aligned with the outer surface of first bone portion 1102 (e.g., as shown at FIG. 11D aligned with the outer medial surface of a first metatarsal bone portion) while respective distal ends 406, 406A are at (e.g., within) second bone portion 1104 (e.g., second portion of first metatarsal) and respective portions of body 402, 402A of screws 400, 400A are across separation 1106.

As noted, the alignment feature 470 at screw 400 can be useful in providing a surface at screw 400 that substantially complements (e.g., substantially corresponds to) one or more predetermined anatomical feature(s) defined at foot 200. For example, the alignment surface 471, at the alignment feature 470, can define a beveled alignment surface that is configured to align with an outer surface of a bone portion, such as an outer medial surface of a first, proximal metatarsal bone portion 1102 (e.g., as shown at FIGS. 11B and 11D). As one such example shown at FIG. 11B, the beveled alignment surface 471 can define a bevel angle 1150 relative to the central longitudinal axis 408 of the screw 400, and the bevel angle 1150 can be configured to cause the beveled alignment surface 471 to align with and interface (e.g., flushly interface) the outer surface of a bone portion, such as an outer medial surface of a first, proximal metatarsal bone portion 1102. As examples, the bevel angle 1150 can range from twenty to eighty degrees, such as ranging from thirty to seventy degrees, ranging from forty to sixty degrees, or ranging from forty to fifty degrees. For instance, as illustrated at FIG. 11B, first, proximal metatarsal bone portion 1102 can extend along proximal metatarsal bone portion longitudinal axis 1153, second, distal metatarsal bone portion 1104 can extend along distal metatarsal bone portion longitudinal axis 1152, and first, proximal metatarsal bone portion 1102 can be offset relative to second, distal metatarsal bone portion 1104 by a metatarsal medial projection angle 1151 defined between the proximal metatarsal bone portion longitudinal axis 1153 and the distal metatarsal bone portion longitudinal axis 1152. The screw 400 can be configured such that the bevel angle 1150 substantially corresponds to the metatarsal medial projection angle 1151. For instance, at FIG. 11B the metatarsal medial projection angle 1151 can be said to be approximately forty degrees and thus the bevel angle 1150 can be approximately forty degree and thereby substantially correspond to the metatarsal medial projection angle 1151.

In some such embodiments, the screw 400 can have the bevel angle 1150 configured relative to the length L of the screw 400. For example, the screw 400 can be configured such that: (i) the length L of the screw 400 causes the distal end 406 of the screw 400 to be implanted within the second, distal first metatarsal bone portion 1104 and causes the proximal end 404 of the screw 400 to be implanted within the first, proximal first metatarsal bone portion 1106 with the bevel angle 1150, and (ii) as a function of such length L of the screw 400 the bevel angle 1150 is configured to cause the beveled alignment surface 471 to align with and interface (e.g., flushly interface) the outer medial surface of the first, proximal metatarsal bone portion 1102. For instance, the length L of the screw 400 can range from 15-75 mm (e.g., range from 20-60 mm, range from 20-50 mm, or range from 30-60 mm) while the bevel angle 1150, configured relative to the length L of the screw 400, can range from forty to sixty degrees (e.g., range from forty to fifty degrees).

As disclosed elsewhere herein, the screw 400 can include a plurality of differently sized lobes (e.g., lobes 430) that are indexed relative to the location of the alignment feature 470 radially (e.g., circumferentially) at the proximal end 404 of the screw 400. This indexed, relative arrangement of the plurality of different sized lobes to the location of the alignment feature 470 can help to position the alignment feature 470 relative to the one or more predetermined anatomical feature(s) defined at foot 200, such to help position the alignment feature 470 relative to an outer medial surface of a first, proximal metatarsal bone portion 1102. This can be particularly useful in instances where the screw 400 is to be implanted at the foot 200 through a relatively small incision (and with the driver engaged and over the incision) that may obstruct direct visualization of the orientation of the alignment feature 470 relative to the outer medial surface of a first, proximal metatarsal bone portion 1102. Accordingly, engaging the screw 400 with a screw-specific driver at these indexed lobes 430 can provide a rotational position reference as to the alignment feature 470 relative to the one or more predetermined anatomical feature(s) defined at foot 200, such as relative to an outer medial surface of a first, proximal metatarsal bone portion 1102.

For example, during a screw implantation procedure, a clinician can use the screw-specific driver engaged at the plurality of differently sized lobes that are indexed relative to the location of the alignment feature at the screw to cause the alignment feature at the screw to align with one or more predetermined anatomical feature(s) defined at foot 200, such as to align the alignment feature with the outer medial surface of the first, proximal metatarsal bone portion 1102 as shown at the example of FIG. 11B. When the screw-specific driver is engaged at the plurality of differently sized lobes that are indexed relative to the location of the alignment feature at the screw, the clinician can rotatably turn the screw-specific driver until the alignment feature at the screw is angularly aligned with medial side face of the metatarsal bone portion. As disclosed elsewhere herein, in various embodiments the plurality of differently sized lobes can only allow the screw-specific driver to be engaged at (e.g., inserted within) the screw at a preset rotational orientation. As a result, with the screw-specific driver engaged at (e.g., inserted within) the screw at the preset rotational orientation relative to the plurality of indexed lobes at the screw, the clinician can rotatably turn the screw-specific driver until the rotational position of the screw-specific driver (e.g., the rotational position of a handle of the screw-specific driver) corresponds to the beveled alignment surface at the screw is angularly aligned with medial side face of the metatarsal bone portion.

In some such embodiments, when the screw-specific driver is coupled to the screw, the screw-specific driver can obstruct a user's ability to visually discern the alignment feature at the screw. For instance, because the screw-specific driver can be coupled to the same end of the screw that includes the alignment feature and a user's angle of visualization during an screw implantation technique can coincide with the axis along which the screw-specific driver is coupled to the screw, a user may not be able to fully, or partially, visually discern the location (e.g., rotational location) of the alignment feature at the screw relative to the predetermined anatomical feature defined at the foot of the patient (e.g., a predetermined anatomical alignment feature associated with a minimally invasive osteosynthesis procedure, such as an outer surface of a metatarsal bone portion of the foot). Accordingly, in some embodiments, to help a user discern (e.g., visually discern, tactilely discern) a rotational location of the alignment feature at the screw relative to the predetermined anatomical feature defined at the foot of the patient, the screw-specific driver can include an alignment feature locational indicator. The alignment feature locational indicator at the screw-specific driver can be configured, when the screw-specific driver is coupled to the screw, to provide an indication (e.g., visual indication, tactile indication) of a rotational location of the alignment feature at the screw relative to the predetermined anatomical feature at the patient to which a user wishes to rotationally align the alignment feature at the screw. For instance, the alignment feature locational indicator can be included at a body of the screw-specific driver, and a user can drive the screw into first and second bone portions until the screw is implanted therein an appropriate depth and the alignment feature locational indicator at the screw-specific driver is aligned (e.g., visually aligned) with the predetermined anatomical feature at the patient to which a user wishes to rotationally align the alignment feature at the screw. Then, when the alignment feature locational indicator at the screw-specific driver is aligned (e.g., visually aligned) with the predetermined anatomical feature at the patient to which a user wishes to rotationally align the alignment feature at the screw, a user can determine that the screw's alignment feature is also aligned (e.g., also rotationally aligned) with the same predetermined anatomical feature at the patient, and thus the user can remove the screw-specific driver from the screw.

Additional details on example instruments and techniques that can be used to implant one or more screws 400 according to the disclosure can be found in U.S. Provisional Patent Application No. 63/778, 198, titled “METATARSAL OSTEOTOMY SYSTEMS AND TECHNIQUES WITH SCREW FIXATION,” filed Mar. 26, 2025, the entire contents of which is incorporated herein by reference.

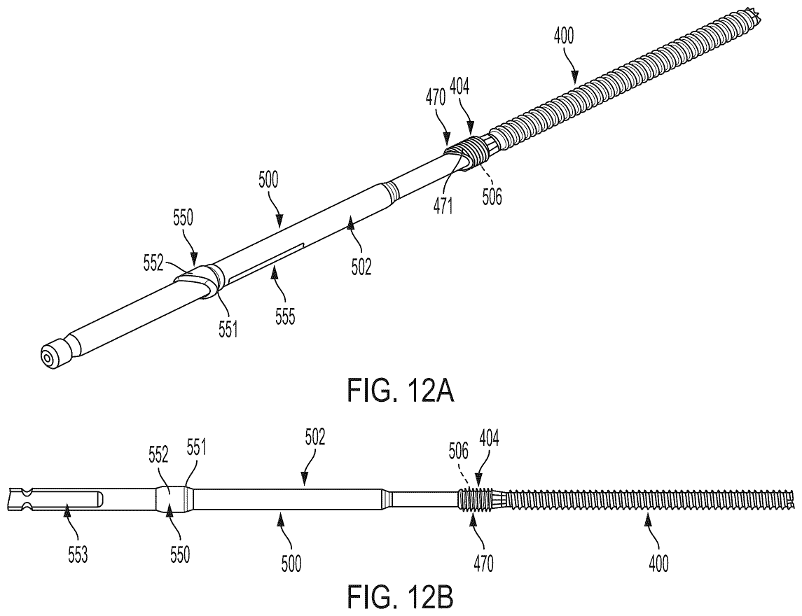

FIGS. 12A and 12B illustrate an embodiment of a screw-specific driver, which includes an alignment feature locational indicator, coupled to a screw, which includes an alignment feature. FIG. 12A shows a perspective view of the screw-specific driver coupled to the screw at a first side, and FIG. 12B shows an elevational view of the screw-specific driver coupled to the screw at a second, different side than that shown at FIG. 12A. As one example shown here, the screw-specific driver at FIGS. 12A and 12B can be the screw-specific driver 500 described previously (e.g., in reference to FIG. 5), and the screw at FIGS. 12A and 12B can be the screw 400 described previously (e.g., in reference to FIGS. 4A-4B).

As noted, screw-specific driver 500 can include alignment feature locational indicator 550. The alignment feature locational indicator 550 at screw-specific driver 500 can be configured to help a user discern (e.g., visually discern, tactilely discern) a rotational location of the alignment feature 470 at the screw 400 relative to the predetermined anatomical feature at the foot of the patient. Namely, the alignment feature locational indicator 550 can include at least a portion that is axially aligned about a perimeter of driver body 502 with alignment feature 470 at screw 400, and thus alignment feature locational indicator 550 can be configured to serve as a proxy for a rotational location of the alignment feature 470 at screw 400 relative to the predetermined anatomical feature at the foot of the patient.

Alignment feature locational indicator 550 can be included at the driver body 502. Alignment feature locational indicator 550 can be included at the driver body 502. In the example shown here, alignment feature locational indictor 550 is a structural element defined at the driver body 502 and protrudes out from the driver body 502 in a direction generally perpendicular to the driver body's central longitudinal axis. In other examples, the alignment feature locational indictor 550 can be a marking or other visual annotation at the driver body 502 and/or the alignment feature locational indictor 550 can include a type of tactile mechanism forming at least part of the alignment feature locational indictor 550. At least a portion of the alignment feature locational indictor 550 can extend around some, but not all, of a perimeter of the driver body 502. For instance, as shown here, alignment feature locational indictor 550 can include a base 551 that can extend around some or all of the perimeter of the driver body 502, a longitudinal base projection 552 that can extend around only a portion, and not all, of the perimeter of the driver body 502, and/or a driver perimeter surface contour 553. The driver perimeter surface contour 553 is shown for the example here as a flat face extending about some, but not all, of the perimeter of driver body 502, though in other examples the driver perimeter surface contour 553 can take the form of other types of different surface contours at driver body 502). The longitudinal base projection 552 and the driver perimeter surface contour 553 can be at a same rotational location about the perimeter of driver body 502 such that the longitudinal base projection 552 and the driver perimeter surface contour 553 are axially aligned along a length of driver body 502.

When the screw-specific driver 500 is coupled to the screw 400, such as shown at FIGS. 12A and 12B, the alignment feature locational indictor 550 can be configured to axially align (e.g., axially align along a co-incident central longitudinal axis of the screw 400 and screw-specific driver 500) with alignment feature 470 at screw 400. For instance, referring to the exemplary embodiment, this can include the longitudinal base projection 552 and/or driver perimeter surface contour 553 axially aligning with the alignment feature 470 when the screw-specific driver 500 is coupled to the screw 400. Axial alignment of the alignment feature locational indictor 550 and the alignment feature 470 can allow the alignment feature locational indictor 550 at the screw-specific driver 500 to serve as a rotational location visual proxy for the alignment feature 470 at the screw 400 relative to the predetermined anatomical feature at the patient to which a user wishes to rotationally align the alignment feature at the screw.

In addition to the alignment feature locational indictor 550, the screw-specific driver 500 shown for the illustrated, exemplary embodiment can include a secondary, offset alignment feature locational indictor 555. The secondary, offset alignment feature locational indicator 555 can be offset from at least some of the alignment feature locational indicator 550 around the perimeter of the driver body 502. For example, the longitudinal base projection 552 of the alignment feature locational indictor 550 and driver perimeter surface contour 553 can be at one location around the perimeter of the driver body 502, while the secondary, offset alignment feature locational indictor 555 can be at a second, different location around the perimeter of the driver body 502. As such, the secondary, offset alignment feature locational indictor 555 can be configured to help a user discern (e.g., visually discern, tactilely discern) a rotational location of the alignment feature 470 at the screw 400 relative to the predetermined anatomical feature at the foot of the patient by providing an indication of the rotational location of the alignment feature 470 as spaced apart from the location of the secondary, offset alignment feature locational indictor 555 and about the perimeter of the driver body 502. In other words, while the alignment feature locational indictor 550 (e.g., longitudinal base projection 552 and/or driver perimeter surface contour 553) can provide an indication as to the rotational location of the alignment feature 470 being the rotational location of the alignment feature locational indicator 550 (e.g., being at the rotational location of the longitudinal base projection 552 and/or driver perimeter surface contour 553), the secondary, offset alignment feature locational indictor 555 can provide an indication as to the rotational location of the alignment feature 470 being at a rotational location that is spaced apart about the perimeter of the driver body 502 from the secondary, offset alignment feature locational indictor 555. With the secondary, offset alignment feature locational indictor 555 being generally located at an opposite side of the driver body perimeter than the alignment feature locational indicator 550, this can provide the user with an indication as to the rotational position of the alignment feature 470, relative to the predetermined anatomical location at the foot, as the driver body 502 is driven three hundred and sixty degrees.

As noted, the alignment feature locational indicator 550 can be configured to help a user discern a rotational location of the alignment feature 470 at the screw 400 relative to the predetermined anatomical feature at the foot of the patient. For instance, when the alignment feature 470 is rotationally offset from the predetermined anatomical feature at the foot of the patient, as in the example at FIG. 11A when screw-specific driver 500 is coupled to screw 400, the alignment feature locational indicator 550 (e.g., longitudinal base projection 552 and/or driver perimeter surface contour 553 of alignment feature locational indicator 550) can also be rotationally offset from the predetermined anatomical feature at the foot at the same rotational offset location at the alignment feature 470. Then, when the screw-specific driver 500 is used to further rotationally drive the screw 400 into the one or more bone portions, the screw 400 can be implanted a depth into the one or more bone portions with the alignment feature 470 rotationally aligned with the predetermined anatomical feature at the foot using the alignment feature locational indicator 550 at the screw-specific driver 500. When the alignment feature locational indicator 550 (e.g., longitudinal base projection 552 and/or driver perimeter surface contour 553 of alignment feature locational indicator 550) at the screw-specific driver 500 is rotationally aligned with the predetermined anatomical feature at the foot, a user can determine that the alignment feature 470 is also rotationally aligned with the predetermined anatomical feature at the foot.

Thus, the user may decide to stop rotationally driving the screw 400 further into the one or more bone portions when the alignment feature locational indicator 550 (e.g., longitudinal base projection 552 and/or driver perimeter surface contour 553 of alignment feature locational indicator 550) at the screw-specific driver 500 is rotationally aligned with the predetermined anatomical feature at the foot. To help facilitate this, the plurality of differently sized lobes at the screw 400 can be indexed relative to the alignment feature 470 at the screw 400 such that, when the screw-specific driver 500 is coupled to the screw 400 via these plurality of differently sized lobes, the alignment feature locational indicator 550 at the screw-specific driver 500 is also indexed relative to the alignment feature 470 at the screw 400 via these plurality of differently sized lobes at the screw 400. In one example application, an embodiment of the technique 1000 can include such one or more actions.

Screw and driver configurations according to the disclosure, including screw 400 and screw-specific driver 500 can have a variety of different sizes and configurations and can be used in a variety of different applications. In various applications, the first bone portion 1102 and second bone portion 1104 screw 400 is configured to be inserted into may be two different bones or different portions of a same bone. The bone portions can be separated by separation 1106 that can be a joint (e.g., in a joint fusion procedure), a fracture (e.g., in a fracture repair procedure), or an osteotomy (e.g., in a minimally invasive osteotomy procedure).

While the size of screw 400 may vary, in some applications the screw may have a length L (FIGS. 4A-4B) less than 100 mm, such as less than 75 mm, less than 60 mm, less than 50 mm, less than 40 mm, less than 30 mm, or less than 20 mm. Additionally or alternatively, screw 400 may have a length L greater than 10 mm, such as greater than 15 mm, greater than 20 mm, greater than 25 mm, greater than 30 mm, greater than 35 mm, greater than 40 mm, greater than 45 mm, or greater than 50 mm. For example, screw 400 may have a length L within a rang from 15 mm to 100 mm, such as from 15 mm to 75 mm, from 16 mm to 70 mm, from 16 mm to 60 mm, from 20 mm to 60 mm, or from 30 mm to 90 mm.

With screw 400 having any one of the foregoing lengths (or yet even different length), the body screw 400 may have a diameter 460 (FIGS. 4A-4B) within a range from 2 mm to 10 mm, such as from 3 mm to 6 mm. As specific examples, the diameter 460 of screw 400 may be 3.0 mm, 3.3 mm, 4.0 mm, 4.5 mm, 5.0 mm, or 5.5 mm. The body of screw 400 may have a constant diameter across its length, or the diameter of the screw may vary across its length (e.g., such that the diameter is different at one location along the length of the screw than at least one other location). When the diameter of screw 400 varies across its length, the diameter can be measured at its location of maximum diameter (excluding the head for screw configurations including a head, where the head can optionally have a larger diameter than the body of the screw).

In different implementations of screw 400 according to the disclosure (used alone or in combination with screw-specific driver 500) the screw can have constant or variable pitch threading. When configured as a constant pitch screw, the screw can utilize a uniform thread spacing along the length of the shaft, resulting in consistent axial advancement per rotation. This design allows for predictable engagement with bone and may be used in applications requiring steady compression or secure fixation without differential displacement between fragments.

In contrast, when configured as a variable pitch screw, the screw can utilize threads that vary in spacing—e.g., decreasing or increasing along the screw shaft—to produce differential movement between the near and far bone cortices upon insertion. This variation can facilitate compression of bone portions without the need for external compression devices, which may be beneficial in fracture or osteotomy procedures.

In some applications, screw 400 is configured as a cortical screw, which is configured for engagement in dense cortical bone. A cortical screw configured may feature a smaller thread height and a finer pitch compared to cancellous screws, which may utilize firm anchorage in the dense outer layers of bone.

The threading on screw 400 can be implemented as a single lead or a double lead. In a single-lead configuration, one continuous helical thread wraps around the screw shaft. Each 360-degree turn of the screw advances it axially by the distance equal to the pitch of the thread. In contrast, in a double-lead configuration, two helical threads run in parallel along the screw shaft, starting at offset points. This effectively doubles the lead-that is, the axial advancement per rotation-compared to a single-lead screw of the same pitch.

Screw 400 can be cannulated or non-cannulated. A cannulated screw can be characterized by a central hollow core or lumen that runs along the length of the screw. This allows the screw to be inserted over a wire, providing enhanced precision in placement, particularly in minimally invasive or percutaneous procedures. By contrast, a non-cannulated screw is solid throughout with uninterrupted material continuity through the core of the screw.

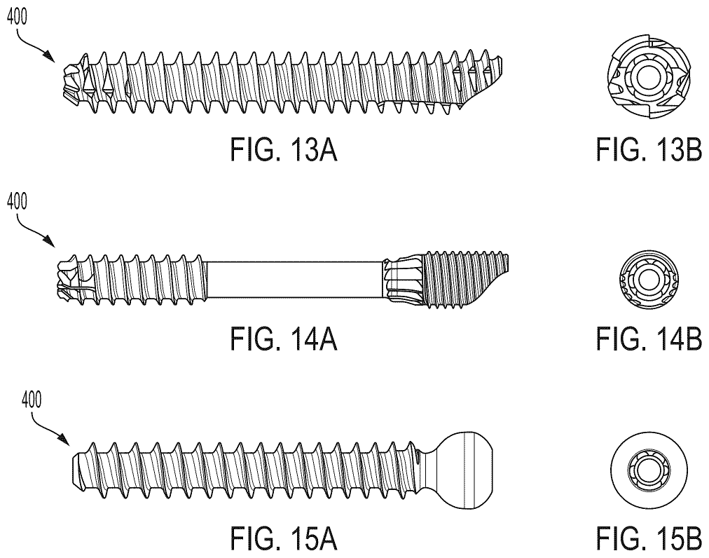

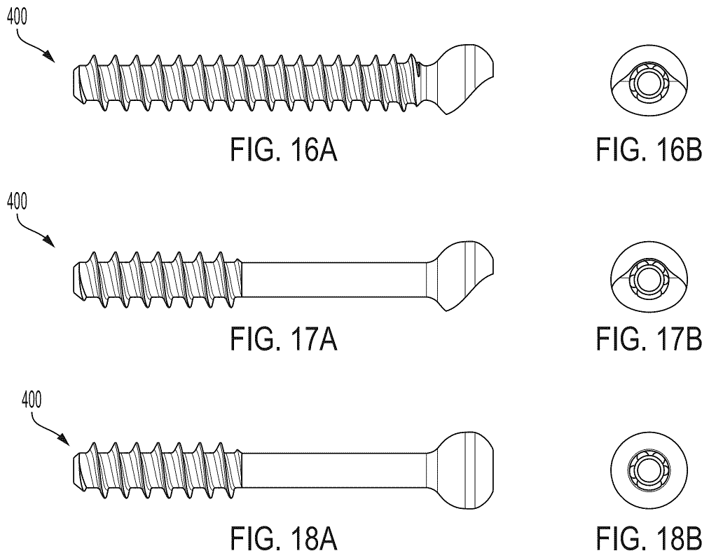

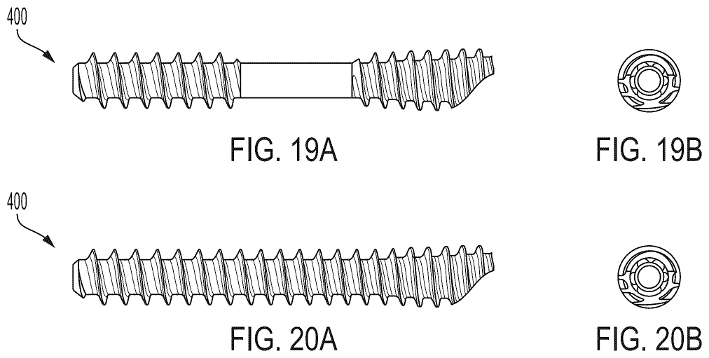

FIGS. 13-20 illustrate different example configurations of screw 400 that can be implemented according to the disclosure. FIGS. 13A and 13B are a side view and an end view, respectively, of an example configuration of screw 400 configured as a variable pitch fully threaded screw with a beveled head. FIGS. 14A and 14B are a side view and an end view, respectively, of an example configuration of screw 400 configured as partially threaded screw for an osteotomy procedure. FIGS. 15A and 15B are a side view and an end view, respectively, of an example configuration of screw 400 configured as variable pitch fully threaded cortical screw. FIGS. 16A and 16B are a side view and an end view, respectively, of an example configuration of screw 400 configured as variable pitch fully threaded cortical screw with a beveled head. FIGS. 17A and 17B are a side view and an end view, respectively, of an example configuration of screw 400 configured as variable pitch partially threaded cortical screw with a beveled head. FIGS. 18A and 18B are a side view and an end view, respectively, of an example configuration of screw 400 configured as variable pitch partially threaded cortical screw. FIGS. 19A and 19B are a side view and an end view, respectively, of an example configuration of screw 400 configured as variable pitch partially threaded headless screw with a beveled head/end. FIGS. 20A and 20B are a side view and an end view, respectively, of an example configuration of screw 400 configured as variable pitch fully threaded headless screw with a beveled head/end.

Any of the instruments and/or implants described herein can be designed and constructed with patient-specific sizing and/or characteristics (e.g., one or more characteristics configured to interface with patient-specific anatomical attributes). In these examples, the anatomical characteristics (e.g., size and/or shape) of at least a portion of the patient's foot undergoing the procedure can be determined prior to performing the surgical procedure. The patient's foot may be imaged to provide data indicative of the size and structure of the patient's foot. A computational model representative of the patient's foot may then be generated and one or more of the instruments and/or implants, such as screw 400, to be used during the procedure sized, shaped, and/or otherwise configured to the specific anatomical characteristics of the foot of the patient undergoing the procedure. The instruments and/or implants can then be manufactured to provide one or more patient-specific components that are then used during the subsequent surgical procedure. For example, the instruments and/or implants may have one or more surface features size and shape indexed to corresponding anatomical location(s) of the patient's bone where the features can be positioned.

Various examples have been described. These and other examples are within the scope of the following claims.

1. A screw for osteosynthesis comprising:
a body comprising a proximal end and a distal end, the body defining a central longitudinal axis that extends between the proximal end and the distal end;
a thread disposed along at least a portion of the body; and
a driver receiving cavity at the proximal end of the body, the driver receiving cavity comprising at least five lobes circumferentially spaced apart from one another about the central longitudinal axis to define at least five grooves,
wherein each one of the at least five lobes defines a different internal lobular area.

2. The screw of claim 1, further comprising an alignment feature disposed along at least a portion of the body, wherein the at least five lobes are indexed relative to the alignment feature.

3. The screw of claim 2, wherein the body has a circular cross-sectional shape, and the alignment feature comprises a bevel extending along at least a portion of a length of the body.

4. The screw of claim 1, wherein the at least five grooves are configured to receive six lobes of a hexalobular driver having equally spaced lobes.

5. The screw of claim 4, wherein one of the at least five grooves is configured to receive two lobes of the hexalobular driver.

6. The screw of claim 4, wherein:
one or more of the at least five lobes is configured to engage the hexalobular driver in a first hexalobular driver rotational direction corresponding to removal of the screw; and
one or more of the at least five lobes is configured to engage the hexalobular driver in a second hexalobular driver rotational direction corresponding to advancement of the screw,
the second hexalobular driver rotational direction being opposite the first hexalobular driver rotational direction.

7. The screw of claim 6, wherein:
at least two of the at least five lobes is configured to engage the hexalobular driver in the first hexalobular driver rotational direction; and
at least two of the at least five lobes is configured to engage the hexalobular driver in the second hexalobular driver rotational direction.

8. The screw of claim 6, wherein a different number of the at least five lobes is configured to engage the hexalobular driver in the first hexalobular driver rotational direction than in the second hexalobular driver rotational direction.

9. The screw of claim 1, wherein each of the at least five grooves defines a different area than each other of the at least five grooves.

10. The screw of claim 1, wherein:
the at least five lobes is only five lobes; and
the at least five grooves is only five grooves.

11. The screw of claim 1, wherein the thread disposed along at least a portion of the body comprises a first threaded region configured to be inserted into a first bone portion and a second threaded region configured to be inserted into a second bone portion, the first threaded region being separated from the second threaded region by a transition region defining at least one cutting feature.

12. The screw of claim 1, wherein:
the driver receiving cavity comprises a driver receptacle that extends from the proximal end of the body toward the distal end of the body;
the at least five lobes are spaced distally from the proximal end; and
each of the at least five lobes comprises at least two lobular sidewalls that protrude radially inwardly into the driver receptacle.

13. The screw of claim 12, further comprising a driver nub receiving bore defined at the body and extending along the central longitudinal axis, wherein:
the driver nub receiving bore is spaced distally from the proximal end and distally from the at least five lobes;
the driver nub receiving bore defines a first internal body diameter transverse to the central longitudinal axis,
each of the at least five lobes defines a second internal body diameter transverse to the central longitudinal axis; and
the first internal body diameter is smaller than the second internal body diameter.

14. The screw of claim 1, wherein the body has a length extending from the proximal end to the distal end sized to be positioned across two portions of a metatarsal bone.

15. A system comprising:
an osteosynthesis screw comprising:
a body comprising a proximal end and a distal end, the body defining a screw central longitudinal axis that extends between the proximal end and the distal end,
a thread disposed along at least a portion of the body, and
a driver receiving cavity at the proximal end of the body, the driver receiving cavity comprising at least three lobes circumferentially spaced apart from one another about the screw central longitudinal axis to define at least three grooves, wherein each one of the at least three lobes defines a different internal lobular area; and
a screw-specific driver comprising:
a driver body comprising a proximal end and a distal end, the driver body defining a driver central longitudinal axis, and
at least three driving lobes at the driver body, the at least three driving lobes being circumferentially spaced apart from one another about the driver central longitudinal axis, wherein each one of the at least three driving lobes defines a different driving lobular area, and each one of the at least three different driving lobular areas correspond to one of the at least three grooves.

16. The system of claim 15, wherein:
the osteosynthesis screw further comprises a driver nub receiving bore defined at the body and extending along the driver central longitudinal axis, the driver nub receiving bore is spaced distally from the proximal end and distally from the at least three lobes, and the driver nub receiving bore defines a first internal body diameter transverse to the driver central longitudinal axis; and
the screw-specific driver further comprises a driver engagement nub at the distal end of the driver body, and the at least three driving lobes are spaced proximally from the driver engagement nub such that the driver engagement nub is configured to engage the osteosynthesis screw prior to the at least three driving lobes engaging the osteosynthesis screw.

17. The system of claim 15, wherein the osteosynthesis screw further comprises an alignment feature disposed along at least a portion of the body, wherein the at least three lobes are indexed relative to the alignment feature.

18. The system of 17, wherein the body has a circular cross-sectional shape, and the alignment feature comprises a bevel extending along at least a portion of a length of the body.

19. The system of claim 17, wherein:
the screw-specific driver further comprises an alignment feature locational indicator, and
when the screw-specific driver is coupled to the osteosynthesis screw, the alignment feature locational indicator is axially aligned with the alignment feature at the osteosynthesis screw.

20. The system of claim 19, wherein:
the at least three lobes at the driver receiving cavity are indexed relative to the alignment feature, and
when the screw-specific driver is coupled to the osteosynthesis screw, the alignment feature locational indicator is indexed relative to the alignment feature.

21. The system of claim 15, wherein the at least three grooves are configured to receive a hexalobular driver having equally spaced lobes, and of the at least three grooves is configured to receive two lobes of the hexalobular driver.

22. A bone fixation technique comprising:
rotationally driving an osteosynthesis screw attached to a screw-specific driver through a first bone portion and into a second bone portion and across a separation between the first bone portion and the second bone portion,
wherein the screw-specific driver is engaged with a driver receiving cavity of the osteosynthesis screw, and the driver receiving cavity comprises at least three lobes circumferentially spaced apart from one another about a screw central longitudinal axis to define at least three grooves, each one of the at least three lobes defines a different internal lobular area.

23. The bone fixation technique of claim 22, further comprising cutting a metatarsal bone into the first bone portion and the second bone portion.
